(19) United States
(12) Patent Application Publication
(10) Pub. No.: US 2025/0091998 A1
(43) Pub. Date: Mar. 20, 2025

(54) A PROCESS FOR THE PREPARATION OF CARBAMOYL BENZAMIDE PHENYL ISOXAZOLINE CLASS DRUG/S AND ITS INTERMEDIATES

(71) Applicant: ZENFOLD SUSTAINABLE TECHNOLOGIES PRIVATE LIMITED, Bangalore (IN)

(72) Inventors: Phaneendrasai Karri, Guntur (IN); Srinivasarao Balla, Bengaluru (IN)

(73) Assignee: ZENFOLD SUSTAINABLE TECHNOLOGIES PRIVATE LIMITED, Bangalore (IN)

(21) Appl. No.: 18/294,170

(22) PCT Filed: Jul. 31, 2022
(86) PCT No.: PCT/IN2022/050690

§ 371 Date: Feb. 1, 2024

(30) Foreign Application Priority Data
Aug. 1, 2021 (IN) 202141034601

(51) Int. Cl. C07D 261/04; C07D 413/04

(52) U.S. Cl. CPC C07D 261/04; C07D 413/04

(57) ABSTRACT

The present invention provides a process for the preparation of a compound of Formula I,

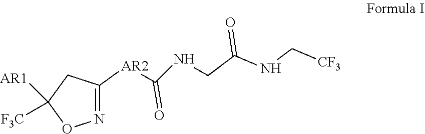

wherein,
AR1 is

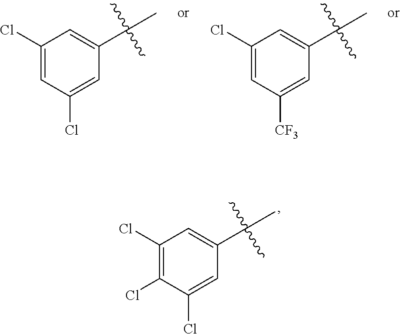 [SMILES: *c1cc(Cl)cc(Cl)c1 ; *c1cc(Cl)cc(C(F)(F)F)c1 ; *c1cc(Cl)c(Cl)c(Cl)c1]

and
AR2 is

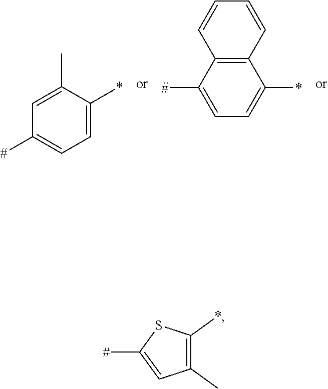

#indicates the attachment to isoxazoline ring and * indicates the attachment to the carbonyl carbon,

The present invention particularly provides the process for the preparation of Fluralaner, Afoxolaner or Lotilaner, which is high yielding, which gives high purity, and which is environment-friendly.

### FIELD OF THE INVENTION

The present invention relates to a process for the preparation of a drug from the isoxazoline class of parasiticides and its intermediates.

The present invention, particularly, relates to a process for the preparation of laners of isoxazoline class of parasiticides and intermediates thereof.

### BACKGROUND OF THE INVENTION

Isoxazolines are a novel class of parasiticides that are potent inhibitors of γ-aminobutyric acid (GABA)-gated chloride channels (GABACIs) and L-glutamate-gated chloride channels (GluCls). Isoxazolines with insecticidal and tickicidal efficacy are non-competitive GABA (gamma-aminobutyric acid) receptor antagonists, much more selective for GABA receptors in insects or ticks, than for those in mammals, including humans. They bind to chloride channels in nerve and muscle cells, which blocks the transmission of neuronal signals. Affected parasites are paralyzed and die. They have a broad spectrum of insecticidal and acaricidal activity and are effective against a number of veterinary parasites such as fleas and ticks.

Fluralaner i.e., 4-[5-(3,5-dichlorophenyl)-5-(trifluoromethyl)-4H-1,2-oxazol-3-yl]-2-methyl-N-[2-oxo-2-(2,2,2-trifluoroethylamino) ethyl] benzamidehas good selectivity than other classes against insect and parasite species and blocks homo-oligomeric GABA receptors expressed in cell lines with high potency.

Another, compound belongs to the isoxazoline chemical compound group is Afoxolaner which is an insecticide and acaricide. It acts as an antagonist at ligand-gated chloride channels, in particular those gated by the neurotransmitter gamma-aminobutyric acid (GABA-receptors). Isoxazolines, among the chloride channel modulators, bind to a distinct and unique target site within the insect GABA-gated chloride channels, thereby blocking pre- and post-synaptic transfer of chloride ions across cell membranes.

Still another, compound belongs to the isoxazoline chemical compound group is Lotilaner which a veterinary drug used to control fleas and ticks in dogs. It is indicated for the treatment and prevention of flea infestations (Ctenocephalides felis) and for the treatment and control of tick infestations including lone star tick (Amblyomma americanum), American dog tick (Dermacentor variabilis), black-legged tick (Ixodes scapularis), and brown dog tick.

The preparation of laners such as fluralaner has been disclosed in various prior art documents. For instance, WO2010005048A1 discloses a process for the preparation of fluralaner which comprises the following steps:
(a) reacting a an isoxazoline-substituted benzene compound of Formula Formula (3) with a 2-aminoacetic acid amide compound of Formula (2),

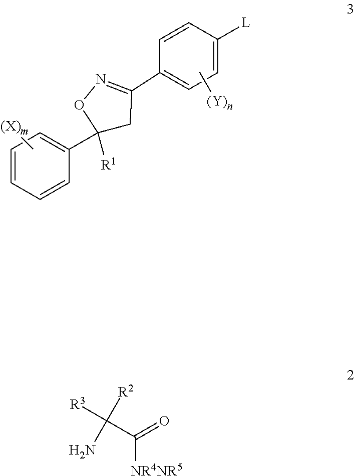

in the presence of carbon monoxide and a palladium catalyst or a base or a condensing agent to obtain a compound of Formula (1); and

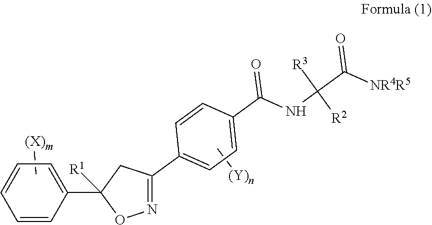

(b) converting the compound of Formula (1) into a compound of Formula (1-1).

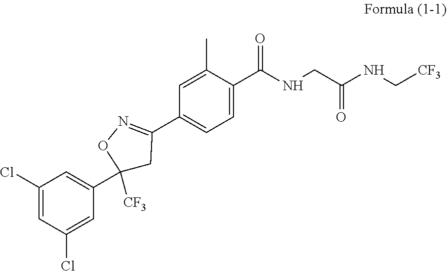 [SMILES: Cc1cc(C2=NOC(c3cc(Cl)cc(Cl)c3)(C(F)(F)F)C2)ccc1C(=O)NCC(=O)NCC(F)(F)F]

Further, WO2009126668 discloses a process for preparing afoxolaner. In the process, 3-trifluoromethyl chalcone of 1 is prepared as shown in the scheme below:

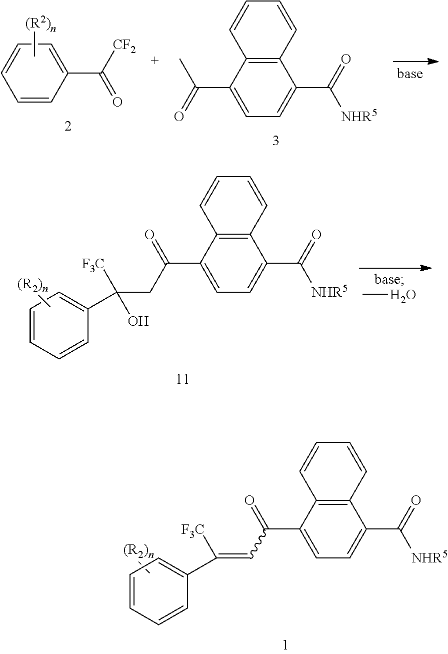

The chalcone 1 is cyclized into corresponding isoxazoline 7d

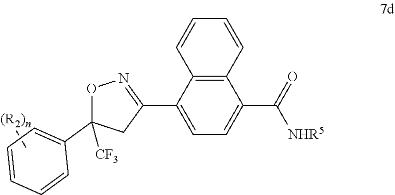

and then converted into Afoxolaner.

WO2009126668 also discloses the preparation of the corresponding isoxazoline 7d by two ways as below:

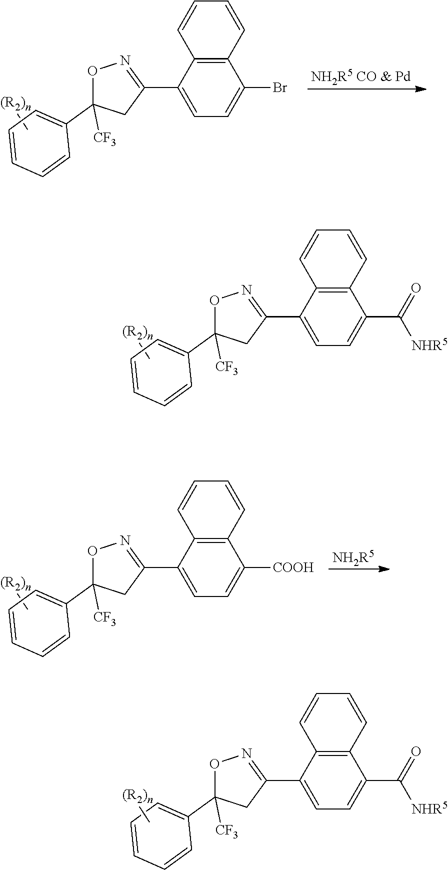

Furthermore, WO2014090918 discloses a process for the preparation of Lotilaner. The process steps comprise cyclizing a compound of Formula III

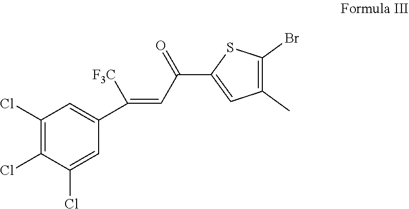 [SMILES: Cc1cc(C(=O)/C=C(/c2cc(Cl)c(Cl)c(Cl)c2)C(F)(F)F)sc1Br]

into the isoxazolinebromothiophene

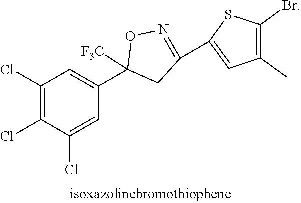 [SMILES: Cc1cc(C2=NOC(c3cc(Cl)c(Cl)c(Cl)c3)(C(F)(F)F)C2)sc1Br]

The isoxazolinebromothiophene is converted into the compound of Formula II

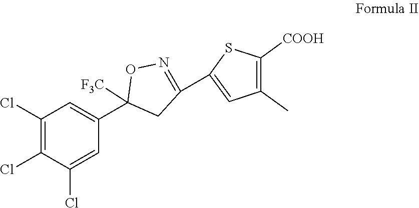 [SMILES: Cc1cc(C2=NOC(c3cc(Cl)c(Cl)c(Cl)c3)(C(F)(F)F)C2)sc1C(=O)O]

The compound of Formula II is then reacted with 2-amino-2′,2′,2′-trifluoroethyl-acetamide hydrochloride to obtain Lotilaner.

Though various prior art patent documents disclose the preparation of Fluralaner, Afoxolaner or Lotilaner, there is still need of a method of synthesizing laner compound which simple, economic, environment-friendly and high yielding.

### OBJECTS OF THE INVENTION

An object of the present invention is to provide a process for the preparation of Fluralaner, Afoxolaner or Lotilaner, which is high yielding.

Another object of the present invention is to a process for the preparation of Fluralaner, Afoxolaner or Lotilaner, which gives high purity.

Still another object of the present invention is to provide a process for the preparation of Fluralaner, Afoxolaner or Lotilaner, which is environment-friendly.

### SUMMARY OF THE INVENTION

The present invention provides a process for the preparation of a compound of Formula I,

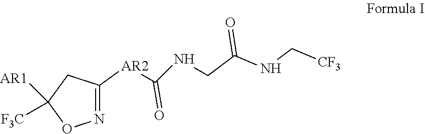

### DESCRIPTION OF THE INVENTION

Accordingly, the present invention provides a process for the preparation of a compound of Formula I,

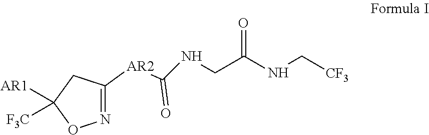

wherein,
AR1 is

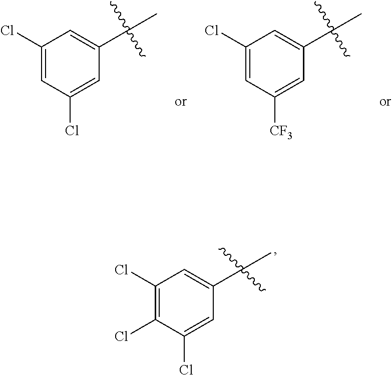 [SMILES: *c1cc(Cl)cc(Cl)c1 ; *c1cc(Cl)cc(C(F)(F)F)c1 ; *c1cc(Cl)c(Cl)c(Cl)c1]

and
AR2 is

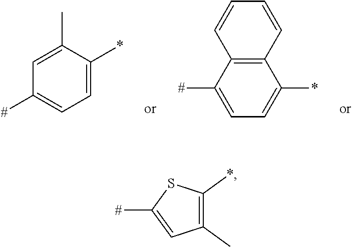

#indicates the attachment to isoxazoline ring and * indicates the attachment to the carbonyl carbon,
said process comprising the following steps:
a) reacting a compound of Formula II

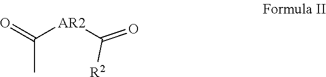

AR2 is

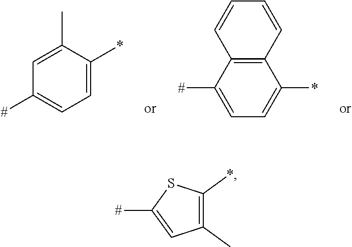

#indicates the attachment to isoxazoline ring and * indicates the attachment to the carbonyl carbon, and
R² is OH, F, Cl or OR³ & R³ is straight or branched chain C₁-C₄ alkyl, with a compound of Formula III

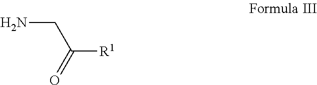

wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
to obtain a compound of Formula IV;

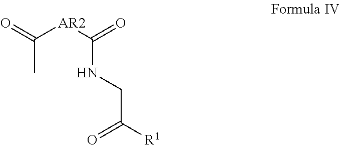

wherein,
AR2 is

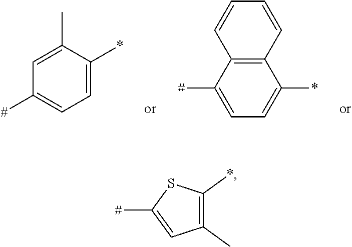

#indicates the attachment to isoxazoline ring and * indicates the attachment to the carbonyl carbon,
R¹ is selected from the group consisting of —OH, —OMe, —OEt, —O(n)Pr, —O(i)Pr, —O(n)Bu, —O(i)Bu, —O(sec)Bu, and —O(tert)Bu,
b) reacting the compound of Formula IV with a compound of Formula V

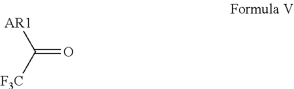

wherein,
AR1 is

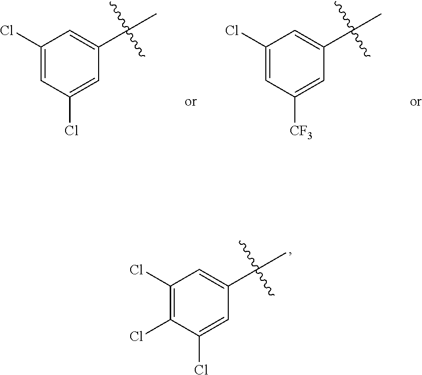 [SMILES: *c1cc(Cl)cc(Cl)c1 ; *c1cc(Cl)cc(C(F)(F)F)c1 ; *c1cc(Cl)c(Cl)c(Cl)c1]

to obtain a compound of Formula VI(i) which is converted to a compound of Formula VI(ii);

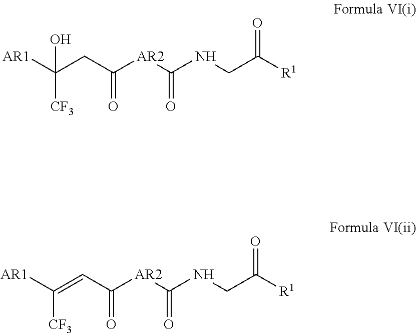

wherein,
AR1 is

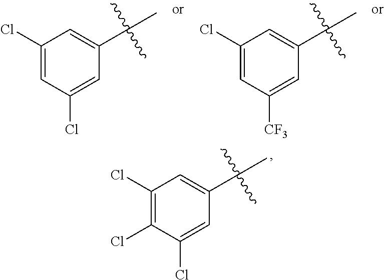 [SMILES: *c1cc(Cl)cc(Cl)c1 ; *c1cc(Cl)cc(C(F)(F)F)c1 ; *c1cc(Cl)c(Cl)c(Cl)c1]

AR2 is

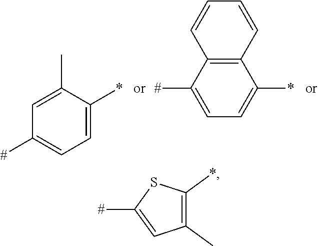

#indicates the attachment to isoxazoline ring and * indicates the attachment to the carbonyl carbon, and
R¹ is selected from the group consisting of —OH, —OMe, —OEt, —O(n)Pr, —O(i)Pr, —O(n)Bu, —O(i)Bu, —O(sec)Bu, and —O(tert)Bu,
c) converting the compound of Formula VI(ii) into a compound of Formula VII;

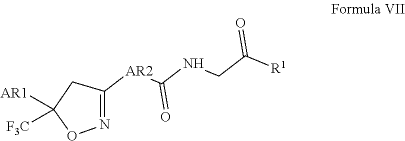

wherein,
AR1 is

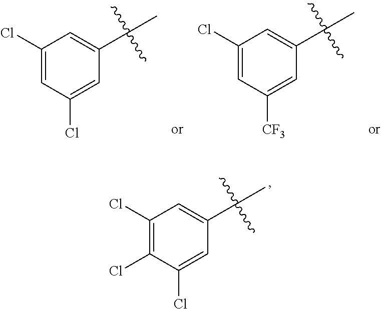 [SMILES: *c1cc(Cl)cc(Cl)c1 ; *c1cc(Cl)cc(C(F)(F)F)c1 ; *c1cc(Cl)c(Cl)c(Cl)c1]

AR2 is

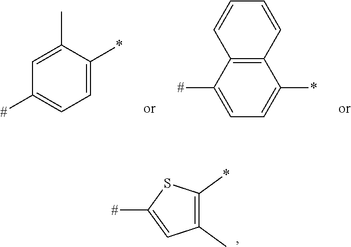

#indicates the attachment to isoxazoline ring and * indicates the attachment to the carbonyl carbon, and
R¹ is selected from the group consisting of —OH, —OMe, —OEt, —O(n)Pr, —O(i)Pr, —O(n)Bu, —O(i)Bu, —O(sec)Bu, and —O(tert)Bu,
d) converting the compound of Formula VII into a compound of Formula VIII(i);

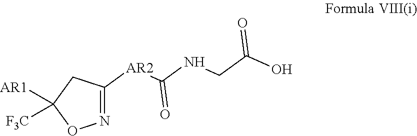

wherein,
AR1 is

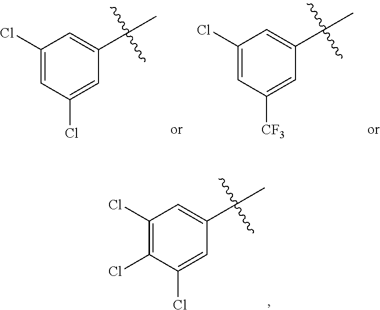 [SMILES: *c1cc(Cl)cc(Cl)c1 ; *c1cc(Cl)cc(C(F)(F)F)c1 ; *c1cc(Cl)c(Cl)c(Cl)c1]

and
AR2 is

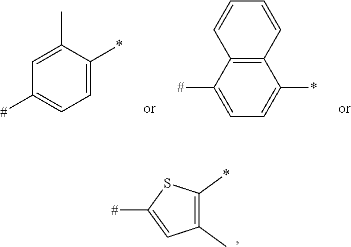

#indicates the attachment to isoxazoline ring and * indicates the attachment to the carbonyl carbon,
e) optionally, converting the compound of Formula VIII(i) into a compound of Formula VIII(ii); and

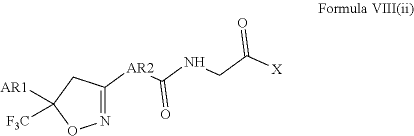

wherein,
AR1 is

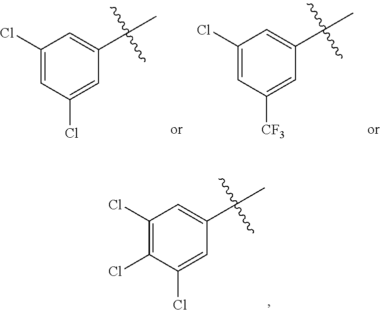 [SMILES: *c1cc(Cl)cc(Cl)c1 ; *c1cc(Cl)cc(C(F)(F)F)c1 ; *c1cc(Cl)c(Cl)c(Cl)c1]

AR2 is

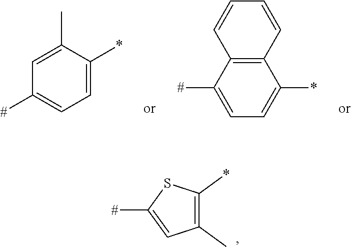

#indicates the attachment to isoxazoline ring and * indicates the attachment to the carbonyl carbon, and
X is Cl or F,
f) reacting the compound of Formula VII or VIII(i) or VIII(ii) with a compound of Formula IX

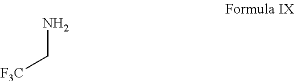

to obtain the compound of Formula I.

In an alternate embodiment, the compound of Formula I is obtained by a process comprising the steps of:
a) reacting the compound of Formula IV

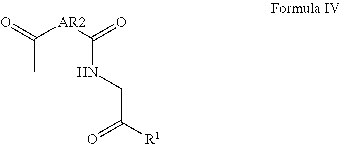

wherein,
AR2 is

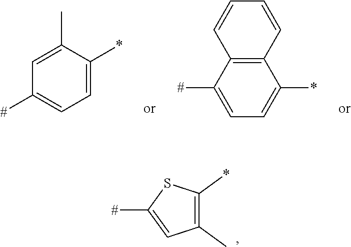

#indicates the attachment to isoxazoline ring and * indicates the attachment to the carbonyl carbon,
R¹ is selected from the group consisting of —OH, —OMe, —OEt, —O(n)Pr, —O(i)Pr, —O(n)Bu, —O(i)Bu, —O(sec)Bu, and —O(tert)Bu,
with the compound of Formula IX

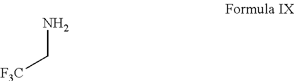

to obtain a compound of Formula X,

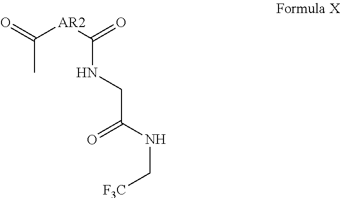

b) reacting the compound of Formula X with the compound of Formula V

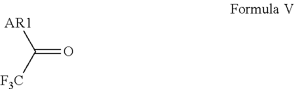

AR1 is

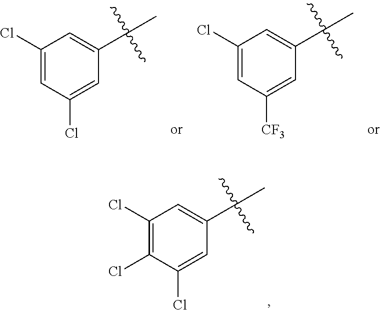 [SMILES: *c1cc(Cl)cc(Cl)c1 ; *c1cc(Cl)cc(C(F)(F)F)c1 ; *c1cc(Cl)c(Cl)c(Cl)c1]

to obtain a compound of Formula XI(i) which is converted to a compound of Formula XI(ii);

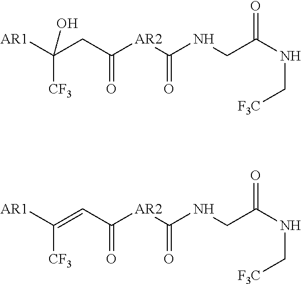

Formula XI(i) Formula XI(ii)
wherein,
AR1 is

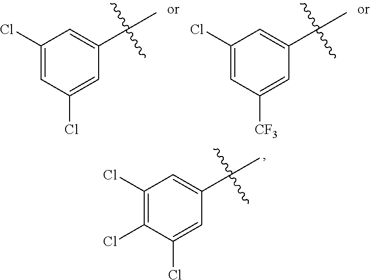 [SMILES: *c1cc(Cl)cc(Cl)c1 ; *c1cc(Cl)cc(C(F)(F)F)c1 ; *c1cc(Cl)c(Cl)c(Cl)c1]

AR2 is

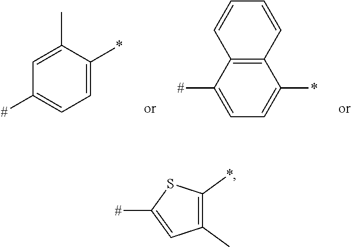

and
#indicates the attachment to isoxazoline ring and * indicates the attachment to the carbonyl carbon,
c) converting the compound of Formula XI(ii) into the compound of Formula I;

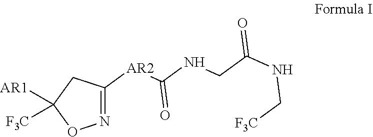

wherein,
AR1 is

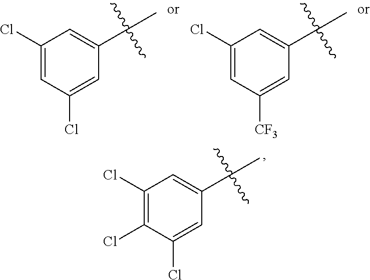 [SMILES: *c1cc(Cl)cc(Cl)c1 ; *c1cc(Cl)cc(C(F)(F)F)c1 ; *c1cc(Cl)c(Cl)c(Cl)c1]

AR2 is

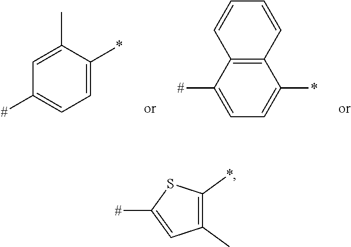

and
#indicates the attachment to isoxazoline ring and * indicates theattachment to the carbonyl carbon.

In one embodiment, any or all of the intermediates of compound of Formula IV, VI, VII, VIII(i) and VIII(ii) formed during the preparation of the compound of Formula I are isolated.

In another embodiment, any or all of the intermediates of compound of Formula IV, VI, VII, VIII(i) and VIII(ii) formed during the preparation of the compound of Formula I are not isolated.

In one embodiment, the compound of Formula II can be prepared from a compound of Formula II-1. The process is described herein after.

### Step II-a:

Acylating the compound of Formula II-1

### AR3-F

### Formula II-1

wherein, AR3 is

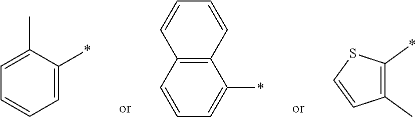

indicates the point of attachment to F atom to obtain a compound of Formula II-2

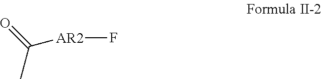

wherein, AR2 is

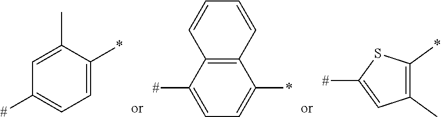

#indicates the attachment to the carbonyl carbon and * indicates the attachment to F atom,

### Step II-b

The compound of Formula II-2 can be converted into a compound of Formula II-3 using a suitable cyano reagent.

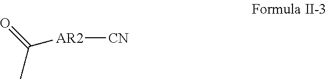

wherein, AR2 is

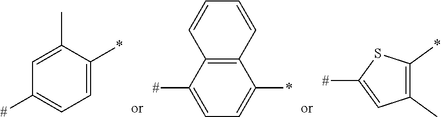

#indicates the attachment to the carbonyl carbon and * indicates the attachment to cyano group,

### Step II-c

The compound of Formula II-3 can be hydrolyzed and optionally, chlorinated using suitable chlorinating agent to obtain the compound of Formula II.

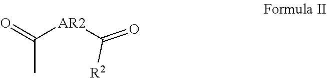

wherein,
AR2 is

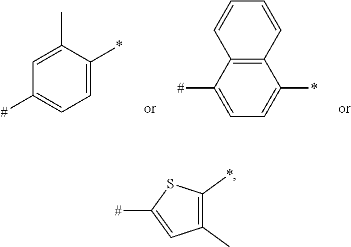

#indicates the attachment to isoxazoline ring and * indicates the attachment to the carbonyl carbon, and
R² is OH, F, Cl or OR³ & R³ is straight or branched chain C₁-C₄ alkyl,

In another aspect, the present invention further provides the compound of Formula IV;

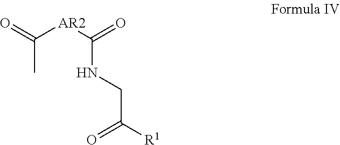

wherein,
AR2 is

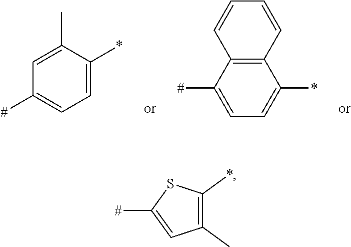

#indicates the attachment to isoxazoline ring and * indicates the attachment to the carbonyl carbon, and
R¹ is selected from the group consisting of —OH, —OMe, —OEt, —O(n)Pr, —O(i)Pr, —O(n)Bu, —O(i)Bu, —O(sec)Bu, and —O(tert)Bu.

In another aspect, the present invention further provides the compound of Formula VI(i) and VI(ii);

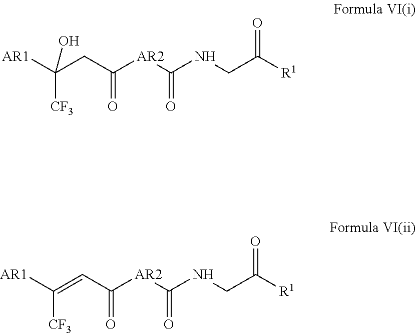

wherein,
AR1 is

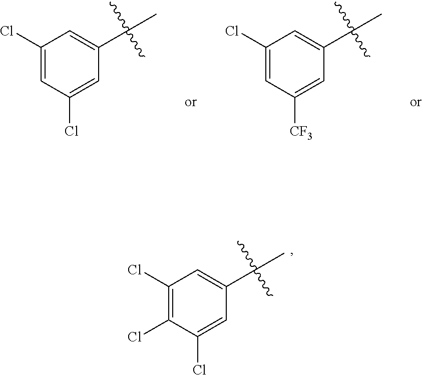 [SMILES: *c1cc(Cl)cc(Cl)c1 ; *c1cc(Cl)cc(C(F)(F)F)c1 ; *c1cc(Cl)c(Cl)c(Cl)c1]

and
AR2 is

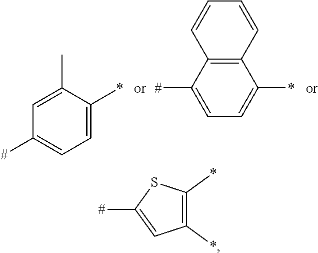

#indicates the attachment to isoxazoline ring and * indicates the attachment to the carbonyl carbon, and
R¹ is selected from the group consisting of —OH, —OMe, —OEt, —O(n)Pr, —O(i)Pr, —O(n)Bu, —O(i)Bu, —O(sec)Bu, and —O(tert)Bu.

The present invention also provides the compound of Formula VII;

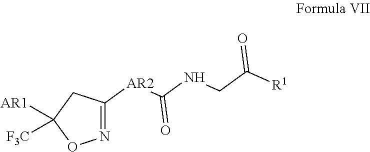

wherein,
AR1 is

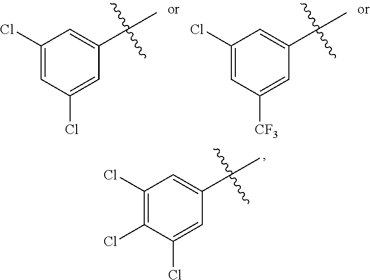 [SMILES: *c1cc(Cl)cc(Cl)c1 ; *c1cc(Cl)cc(C(F)(F)F)c1 ; *c1cc(Cl)c(Cl)c(Cl)c1]

and
AR2 is

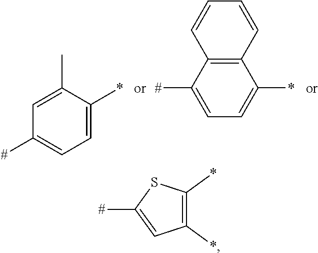

#indicates the attachment to isoxazoline ring and * indicates the attachment to the carbonyl carbon, and
R¹ is selected from the group consisting of —OH, —OMe, —OEt, —O(n)Pr, —O(i)Pr, —O(n)Bu, —O(i)Bu, —O(sec)Bu, and —O(tert)Bu.

In one embodiment, the present invention provides a process for the preparation of compound of Formula I-a (Fluralaner),

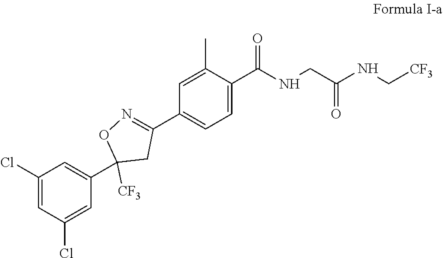 [SMILES: Cc1cc(C2=NOC(c3cc(Cl)cc(Cl)c3)(C(F)(F)F)C2)ccc1C(=O)NCC(=O)NCC(F)(F)F]

said process comprising the following steps:
a) reacting a compound of Formula II-a

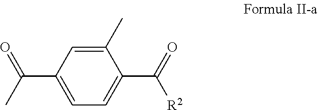

wherein, R² is OH, F, Cl or OR³ & R³ is straight or branched chain C₁-C₄ alkyl, with a compound of Formula III

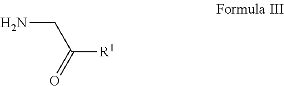

wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
to obtain a compound of Formula IV-a;

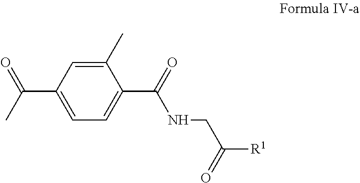

wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
b) reacting the compound of Formula IV-a with a compound of Formula V-a

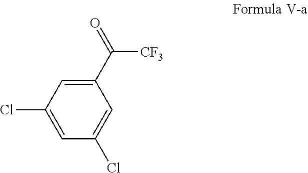 [SMILES: O=C(c1cc(Cl)cc(Cl)c1)C(F)(F)F]

to obtain a compound of Formula VI(i)-a which is converted to a compound of Formula VI(ii)-a;

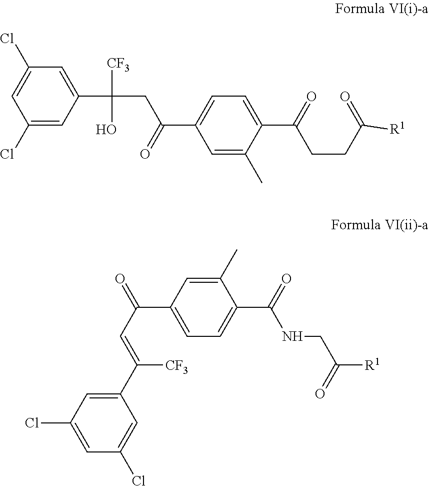

wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
c) converting the compound of Formula VI(ii)-a into a compound of Formula VII-a;

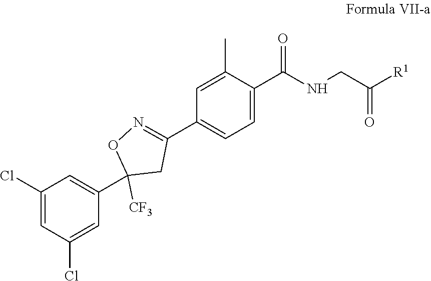

wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
d) converting the compound of Formula VII-a

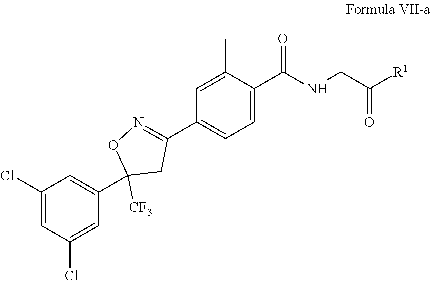

wherein, R¹ is selected from the group consisting of —OMe, —OEt, —O(n)Pr, —O(i)Pr, —O(n)Bu, —O(i)Bu, —O(sec)Bu, and —O(tert)Bu,
into a compound of Formula VIII(i)-a;

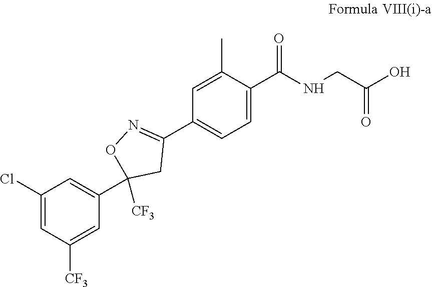 [SMILES: Cc1cc(C2=NOC(c3cc(Cl)cc(C(F)(F)F)c3)(C(F)(F)F)C2)ccc1C(=O)NCC(=O)O]

e) optionally, converting the compound of Formula VIII(i)-a into a compound of Formula VIII(ii)-a; and

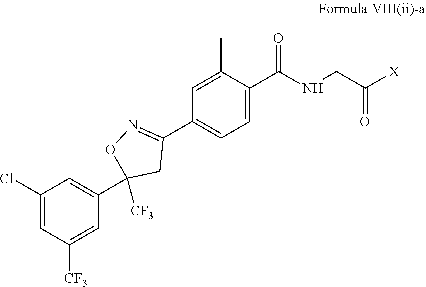

wherein, X is Cl or F,
f) reacting the compound of Formula VII-a or VIII(i)-a or VIII(ii)-a with the compound of Formula IX

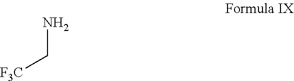

to obtain the compound of formula I-a.

In an alternate embodiment, the compound of Formula I-a is obtained by a process comprising the steps of:
a) reacting the compound of Formula IV-a

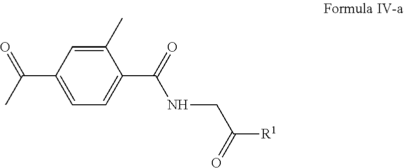

wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
with the compound of Formula IX

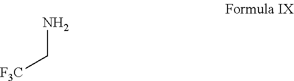

to obtain a compound of Formula X-a,

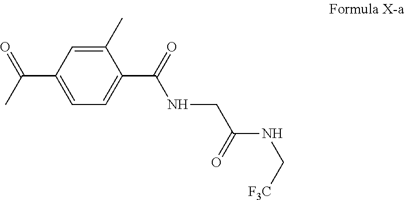 [SMILES: CC(=O)c1ccc(C(=O)NCC(=O)NCC(F)(F)F)c(C)c1]

b) reacting the compound of Formula X-a with the compound of Formula V-a

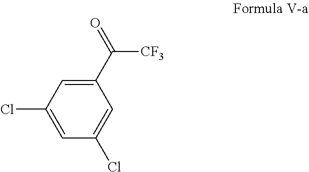 [SMILES: O=C(c1cc(Cl)cc(Cl)c1)C(F)(F)F]

to obtain a compound of Formula XI(i)-a which is converted to a compound of Formula XI(ii)-a;

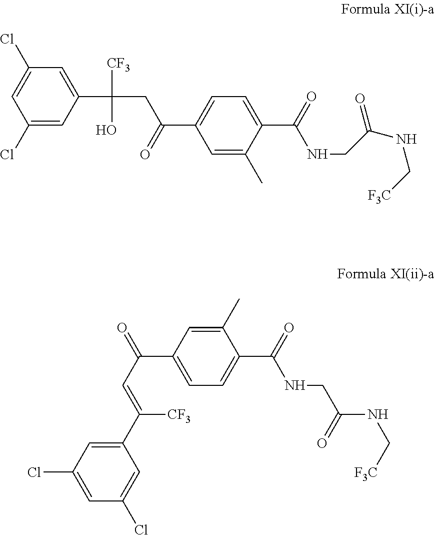 [SMILES: Cc1cc(C(=O)CC(O)(c2cc(Cl)cc(Cl)c2)C(F)(F)F)ccc1C(=O)NCC(=O)NCC(F)(F)F ; Cc1cc(C(=O)/C=C(/c2cc(Cl)cc(Cl)c2)C(F)(F)F)ccc1C(=O)NCC(=O)NCC(F)(F)F]

c) converting the compound of Formula XI(ii)-a into the compound of Formula I-a.

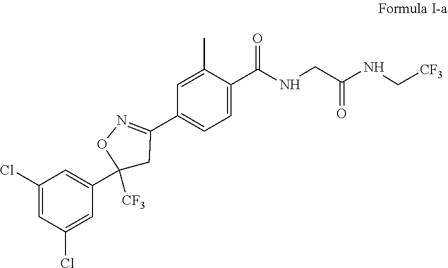 [SMILES: Cc1cc(C2=NOC(c3cc(Cl)cc(Cl)c3)(C(F)(F)F)C2)ccc1C(=O)NCC(=O)NCC(F)(F)F]

In one embodiment, any or all of the intermediates of compound of Formula IV-a, VI-a, VII-a, VIII(i)-a and VIII(ii)-a formed during the preparation of the compound of Formula I are isolated.

In another embodiment, any or all of the intermediates of compound of Formula IV-a, VI-a, VII-a, VIII(i)-a and VIII(ii)-a formed during the preparation of the compound of Formula I are not isolated.

The compound of Formula II-a can be prepared from 2-fluoro toluene. The process is described herein after.

### Step II-a:

Acylating the 2-fluro toluene to obtain a compound of Formula II-1-a.

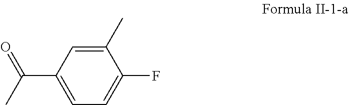

### Step II-b

The compound of Formula II-1-a can be converted into a compound of Formula II-2-a using a suitable cyano reagent.

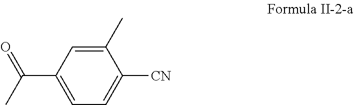

### Step II-c

The compound of Formula II-2-acan be hydrolyzed and optionally, chlorinated using suitable chlorinating agent to obtain the compound of Formula II-a.

In another aspect, the present invention further provides the compound of Formula IV-a;

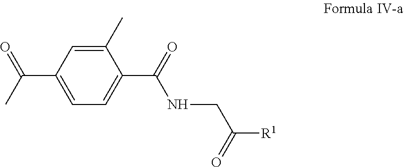

wherein, R¹ is selected from the group consisting of —OH, —OMe, —OEt, —O(n)Pr, —O(i)Pr, —O(n)Bu, —O(i)Bu, —O(sec)Bu, and —O(tert)Bu.

In another aspect, the present invention further provides the compound of Formula VI(i)-a & VI(ii)-a;

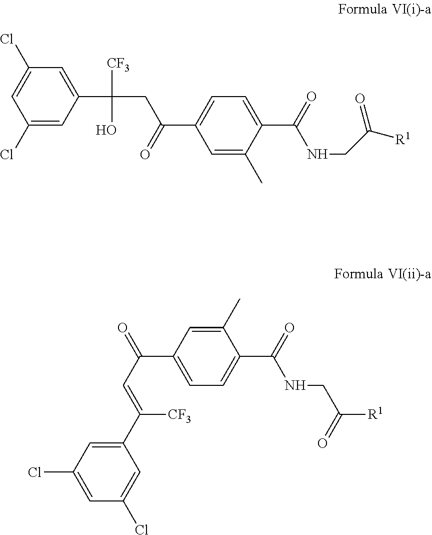

wherein, R¹ is selected from the group consisting of —OH, —OMe, —OEt, —O(n)Pr, —O(i)Pr, —O(n)Bu, —O(i)Bu, —O(sec)Bu, and —O(tert)Bu.

The present invention also provides the compound of Formula VII-a;

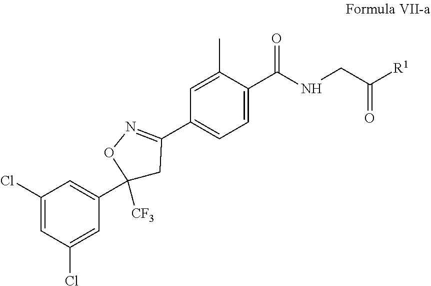

wherein, R¹ is selected from the group consisting of —OEt, —O(n)Pr, —O(i)Pr, —O(n)Bu, —O(i)Bu, —O(sec)Bu, and —O(tert)Bu.

The present invention also provides the compound of Formula XI(i)-a.

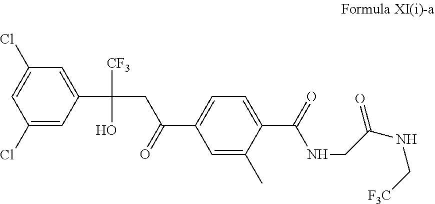 [SMILES: Cc1cc(C(=O)CC(O)(c2cc(Cl)cc(Cl)c2)C(F)(F)F)ccc1C(=O)NCC(=O)NCC(F)(F)F]

In another embodiment, the present invention provides a process for the preparation of compound of Formula I-b (Afoxolaner),

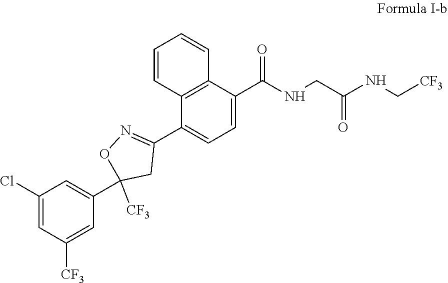 [SMILES: O=C(CNC(=O)c1ccc(C2=NOC(c3cc(Cl)cc(C(F)(F)F)c3)(C(F)(F)F)C2)c2ccccc12)NCC(F)(F)F]

said process comprising the following steps:
a) reacting a compound of Formula II-b

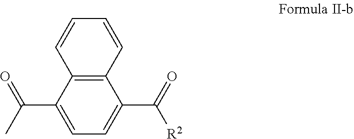

wherein, R² is OH, F, Cl or OR³ & R³ is straight or branched chain C₁-C₄ alkyl, with a compound of Formula III

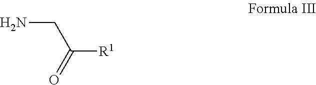

wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
to obtain a compound of Formula IV-b;

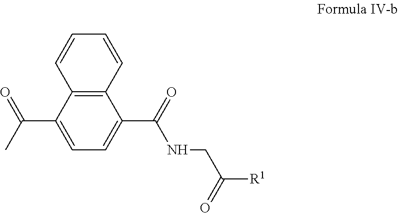

wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
b) reacting the compound of Formula IV-b with a compound of Formula V-b

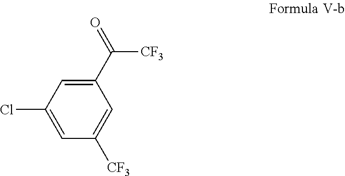 [SMILES: O=C(c1cc(Cl)cc(C(F)(F)F)c1)C(F)(F)F]

to obtain a compound of Formula VI(i)-b which is converted into a compound of Formula VI(ii)-b;

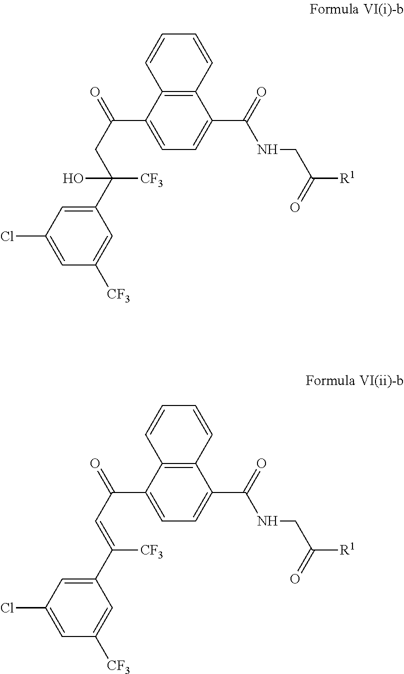

wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
c) converting the compound of Formula VI(ii)-b into a compound of Formula VII-b;

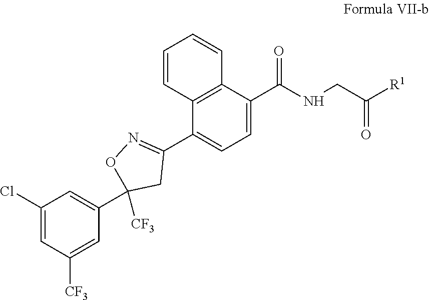

wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
d) converting the compound of Formula VII-b

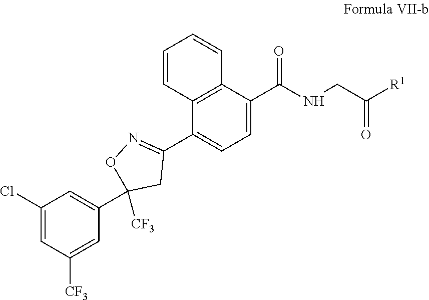

wherein, R¹ is selected from the group consisting of —OMe, —OEt, —O(n)Pr, —O(i)Pr, —O(n)Bu, —O(i)Bu, —O(sec)Bu, and —O(tert)Bu,
into a compound of Formula VIII(i)-b;

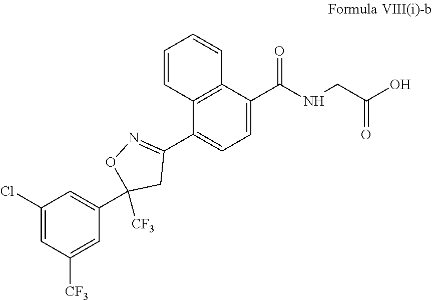 [SMILES: O=C(O)CNC(=O)c1ccc(C2=NOC(c3cc(Cl)cc(C(F)(F)F)c3)(C(F)(F)F)C2)c2ccccc12]

e) optionally, converting the compound of Formula VIII(i)-b into a compound of Formula VIII(ii)-b; and

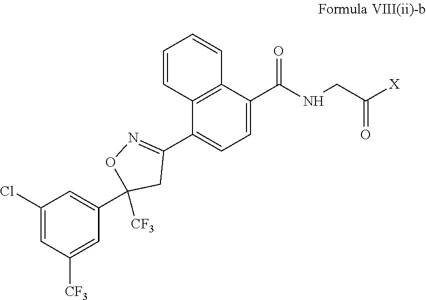

wherein, X is Cl or F,
f) reacting the compound of Formula VII-b or VIII(i)-b or VIII(ii)-b with the compound of Formula IX;

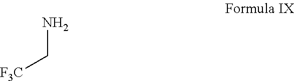

to obtain the compound of formula I-b.

In an alternate embodiment, the compound of Formula I-b is obtained by a process comprising the steps of:
a) reacting the compound of Formula IV-b

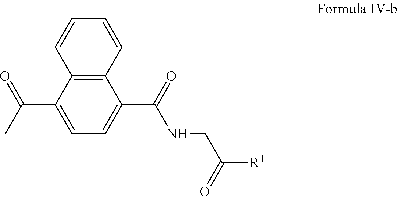

wherein, R¹ is selected from the group consisting of —OH, —OMe, —OEt, —O(n)Pr, —O(i)Pr, —O(n)Bu, —O(i)Bu, —O(sec)Bu, and —O(tert)Bu, with the compound of Formula IX

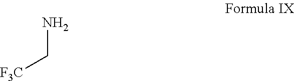

to obtain a compound of Formula X-b,

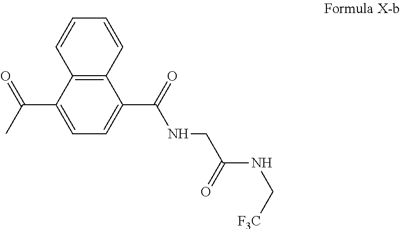 [SMILES: CC(=O)c1ccc(C(=O)NCC(=O)NCC(F)(F)F)c2ccccc12]

b) reacting the compound of Formula X-b with the compound of Formula V-b

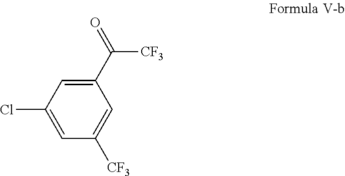 [SMILES: O=C(c1cc(Cl)cc(C(F)(F)F)c1)C(F)(F)F]

to obtain a compound of Formula XI(i)-b which is converted to a compound of Formula XI(ii)-b;

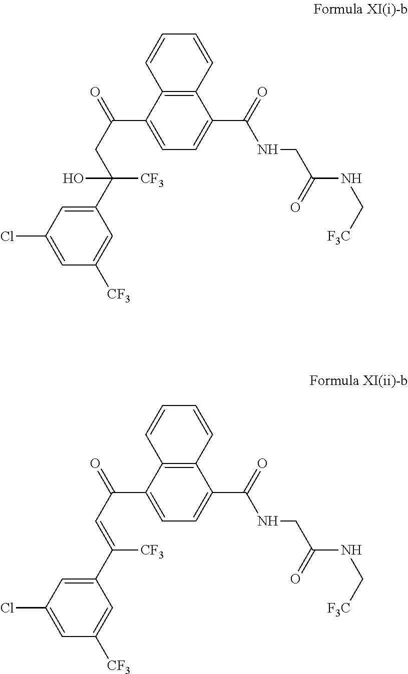 [SMILES: O=C(CNC(=O)c1ccc(C(=O)CC(O)(c2cc(Cl)cc(C(F)(F)F)c2)C(F)(F)F)c2ccccc12)NCC(F)(F)F ; O=C(CNC(=O)c1ccc(C(=O)/C=C(/c2cc(Cl)cc(C(F)(F)F)c2)C(F)(F)F)c2ccccc12)NCC(F)(F)F]

c) converting the compound of Formula XI(ii)-b into the compound of Formula I-b.

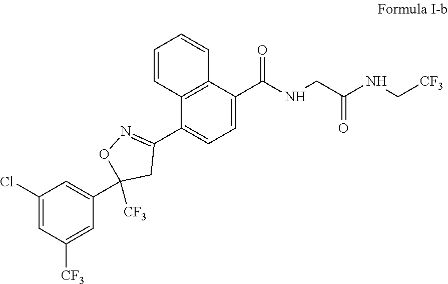 [SMILES: O=C(CNC(=O)c1ccc(C2=NOC(c3cc(Cl)cc(C(F)(F)F)c3)(C(F)(F)F)C2)c2ccccc12)NCC(F)(F)F]

In one embodiment, any or all of the intermediates of compound of Formula IV-b, VI-b, VII-b, VIII(i)-b and VIII(ii)-b formed during the preparation of the compound of Formula I are isolated.

In another embodiment, any or all of the intermediates of compound of Formula IV-b, VI-b, VII-b, VIII(i)-b and VIII(ii)-b formed during the preparation of the compound of Formula I are not isolated.

The compound of Formula II-b can be prepared from 1-fluoro naphthalene. The process is described herein after.

### Step II-a:

Acylating the 1-fluro naphthalene to obtain a compound of Formula II-1-b.

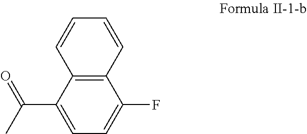

### Step II-b

The compound of Formula II-1-b can be converted into a compound of Formula II-2-b using a suitable cyano reagent.

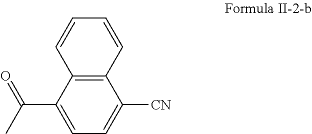

### Step II-c

The compound of Formula II-2-b can be hydrolyzed and optionally, chlorinated using suitable chlorinating agent to obtain the compound of Formula II-b.

In another aspect, the present invention further provides the compound of Formula IV-b;

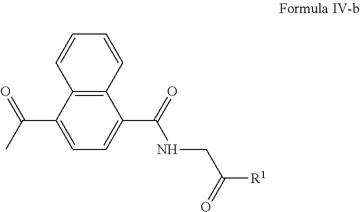

wherein, R¹ is selected from the group consisting of —OH, —OMe, —OEt, —O(n)Pr, —O(i)Pr, —O(n)Bu, —O(i)Bu, —O(sec)Bu, and —O(tert)Bu.

In another aspect, the present invention further provides the compound of Formula VI(i)-b & VI(ii)-b;

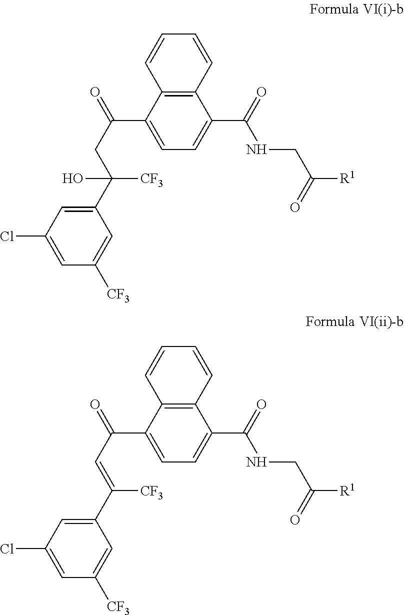

wherein, R¹ is selected from the group consisting of —OH, —OMe, —OEt, —O(n)Pr, —O(i)Pr, —O(n)Bu, —O(i)Bu, —O(sec)Bu, and —O(tert)Bu.

The present invention also provides the compound of Formula VII-b;

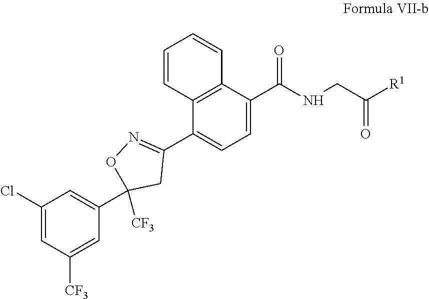

wherein, R¹ is selected from the group consisting of —OH, —OMe, —OEt, —O(n)Pr, —O(i)Pr, —O(n)Bu, —O(i)Bu, —O(sec)Bu, and —O(tert)Bu.

In yet another embodiment, the present invention provides a process for the preparation of compound of Formula I-c (Lotilaner),

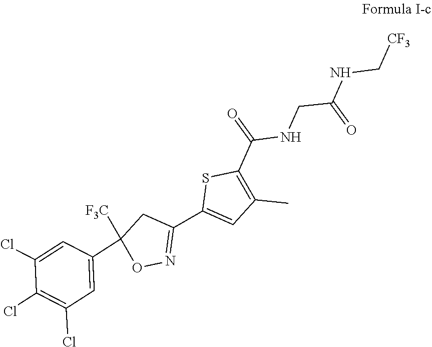 [SMILES: Cc1cc(C2=NOC(c3cc(Cl)c(Cl)c(Cl)c3)(C(F)(F)F)C2)sc1C(=O)NCC(=O)NCC(F)(F)F]

said process comprising the following steps:
a) reacting a compound of Formula II-c

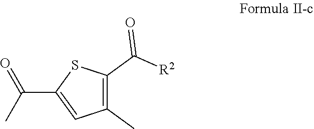

wherein, R² is OH, F, Cl or OR³&R³ is straight or branched chain C₁-C₄ alkyl, with a compound of Formula III

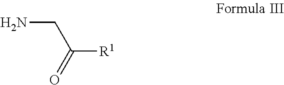

wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
to obtain a compound of Formula IV-c;

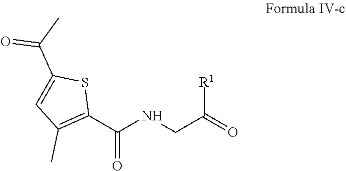

wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
b) reacting the compound of Formula IV-c with a compound of Formula V-c

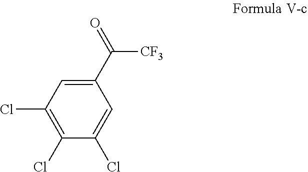 [SMILES: O=C(c1cc(Cl)c(Cl)c(Cl)c1)C(F)(F)F]

to obtain a compound of Formula VI(i)-c which is converted into a compound of Formula VI(ii)-c;

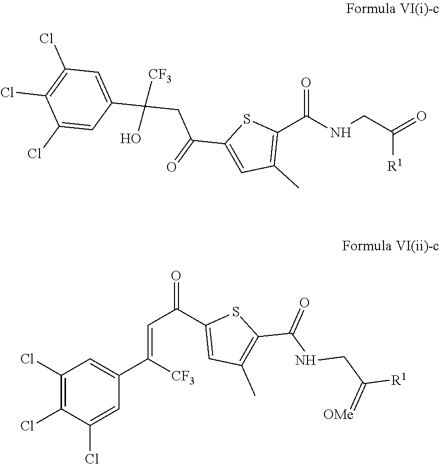

wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
c) converting the compound of Formula VI(ii)-c into a compound of Formula VII-c;

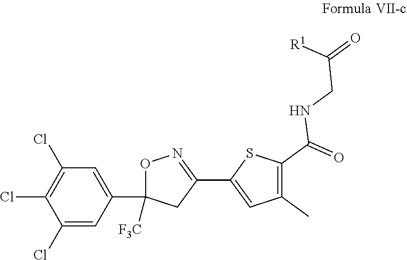

wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
d) converting the compound of Formula VII-c

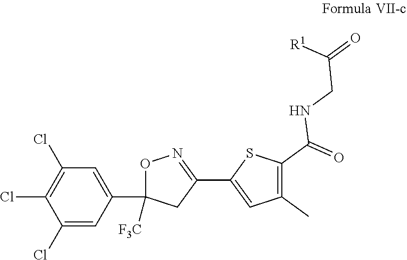

wherein, R¹ is selected from the group consisting of —OMe, —OEt, —O(n)Pr, —O(i)Pr, —O(n)Bu, —O(i)Bu, —O(sec)Bu, and —O(tert)Bu,
into a compound of Formula VIII(i)-c;

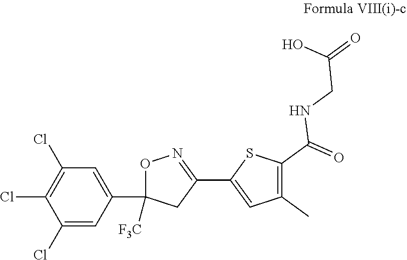 [SMILES: Cc1cc(C2=NOC(c3cc(Cl)c(Cl)c(Cl)c3)(C(F)(F)F)C2)sc1C(=O)NCC(=O)O]

e) optionally, converting the compound of Formula VIII(i)-c into a compound of Formula VIII(ii)-c; and

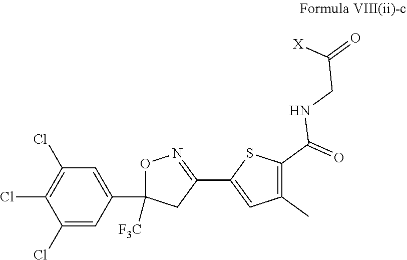

wherein, X is Cl or F,
f) reacting the compound of Formula VII-c or VIII(i)-c or VIII(ii)-c with the compound of Formula IX

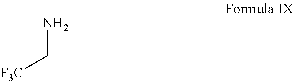

to obtain the compound of formula I-c.

In an alternate embodiment, the compound of Formula I-c is obtained a process comprising the steps of:
a) reacting the compound of Formula IV-c

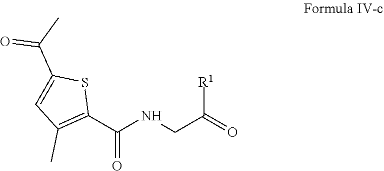

wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
with the compound of Formula IX

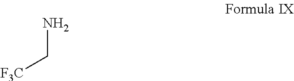

to obtain a compound of Formula X-c,

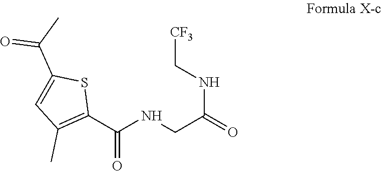 [SMILES: CC(=O)c1cc(C)c(C(=O)NCC(=O)NCC(F)(F)F)s1]

b) reacting the compound of Formula X-c with the compound of Formula V-c

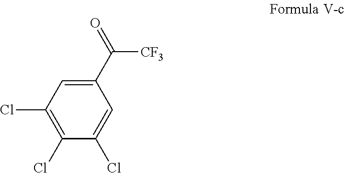 [SMILES: O=C(c1cc(Cl)c(Cl)c(Cl)c1)C(F)(F)F]

to obtain a compound of Formula XI(i)-c which is converted to a compound of Formula XI(ii)-c;

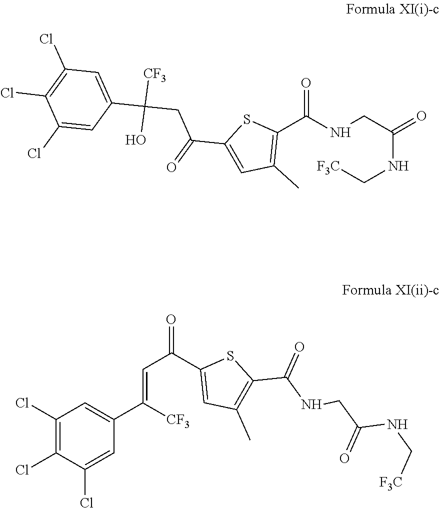 [SMILES: Cc1cc(C(=O)CC(O)(c2cc(Cl)c(Cl)c(Cl)c2)C(F)(F)F)sc1C(=O)NCC(=O)NCC(F)(F)F ; Cc1cc(C(=O)/C=C(/c2cc(Cl)c(Cl)c(Cl)c2)C(F)(F)F)sc1C(=O)NCC(=O)NCC(F)(F)F]

c) converting the compound of Formula XI(ii)-c into the compound of Formula I-c.

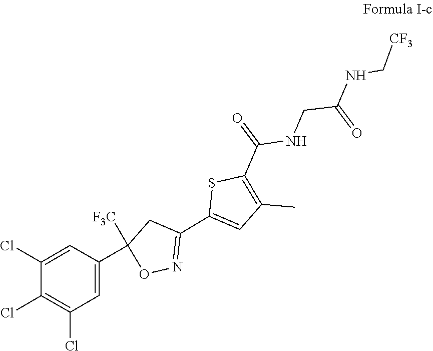 [SMILES: Cc1cc(C2=NOC(c3cc(Cl)c(Cl)c(Cl)c3)(C(F)(F)F)C2)sc1C(=O)NCC(=O)NCC(F)(F)F]

In one embodiment, any or all of the intermediates of compound of Formula IV-c, VI-c, VII-c, VIII(i)-c and VIII(ii)-c formed during the preparation of the compound of Formula I are isolated.

In another embodiment, any or all of the intermediates of compound of Formula IV-c, VI-c, VII-c, VIII(i)-c and VIII(ii)-c formed during the preparation of the compound of Formula I are not isolated.

The compound of Formula II-c can be prepared from 2-fluoro-3-methylthiophene. The process is described herein after.

### Step II-a:

Acylating the 2-fluoro-3-methylthiophene to obtain a compound of Formula II-1-c.

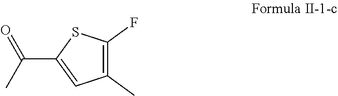

### Step II-b

The compound of Formula II-1-c can be converted into a compound of Formula II-2-c using a suitable cyano reagent.

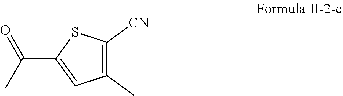

### Step II-c

The compound of Formula II-2-c can be hydrolyzed and optionally, chlorinated using suitable chlorinating agent to obtain the compound of Formula II-c.

In another aspect, the present invention further provides the compound of Formula IV-c;

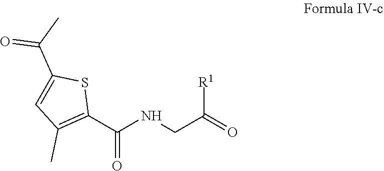

wherein, R¹ is selected from the group consisting of —OH, —OMe, —OEt, —O(n)Pr, —O(i)Pr, —O(n)Bu, —O(i)Bu, —O(sec)Bu, and —O(tert)Bu.

In another aspect, the present invention further provides the compound of Formula VI(i)-c & VI(ii)-c;

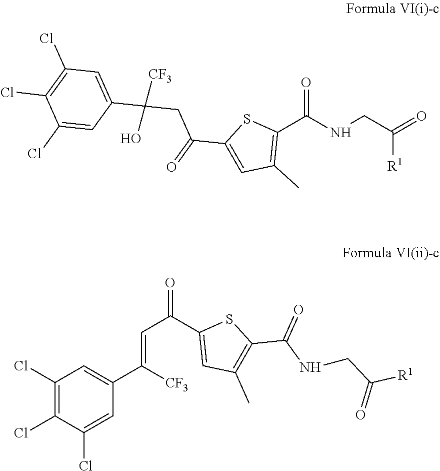

wherein, R¹ is selected from the group consisting of —OH, —OMe, —OEt, —O(n)Pr, —O(i)Pr, —O(n)Bu, —O(i)Bu, —O(sec)Bu, and —O(tert)Bu.

The present invention also provides the compound of Formula VII-c;

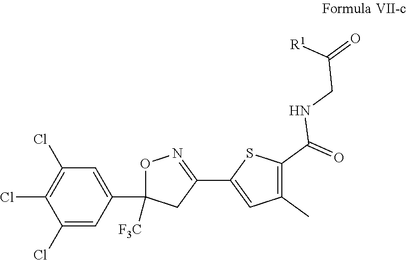

wherein, R¹ is selected from the group consisting of OH, —OEt, —O(n)Pr, —O(i)Pr, —O(n)Bu, —O(i)Bu, —O(sec)Bu, and —O(tert)Bu.

The present invention also provides the compound of Formula X-c.

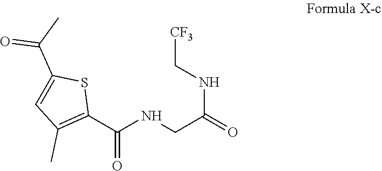 [SMILES: CC(=O)c1cc(C)c(C(=O)NCC(=O)NCC(F)(F)F)s1]

The present invention also provides the compound of Formula XI(i)-c.

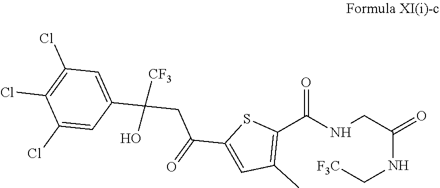 [SMILES: Cc1cc(C(=O)CC(O)(c2cc(Cl)c(Cl)c(Cl)c2)C(F)(F)F)sc1C(=O)NCC(=O)NCC(F)(F)F]

The present invention also provides the compound of Formula XI(ii)-c.

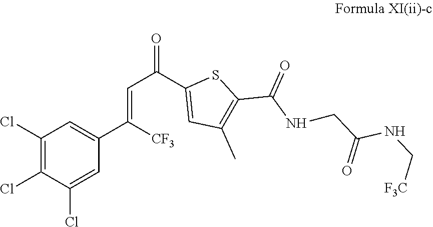 [SMILES: Cc1cc(C(=O)/C=C(/c2cc(Cl)c(Cl)c(Cl)c2)C(F)(F)F)sc1C(=O)NCC(=O)NCC(F)(F)F]

The present invention shall now be described with the help of the non-limiting examples. The solvents, reagents, catalysts, temperature pressure conditions, work up mechanism, mode of addition are merely for illustrative purpose. A person skilled in the art can modify, extrapolate or design around the experiments to achieve the results intended by the present invention. Any modifications, extrapolation or any design around shall be the part of the present invention.

### Example 1: Preparation of Fluralaner (Compound of Formula I-a)

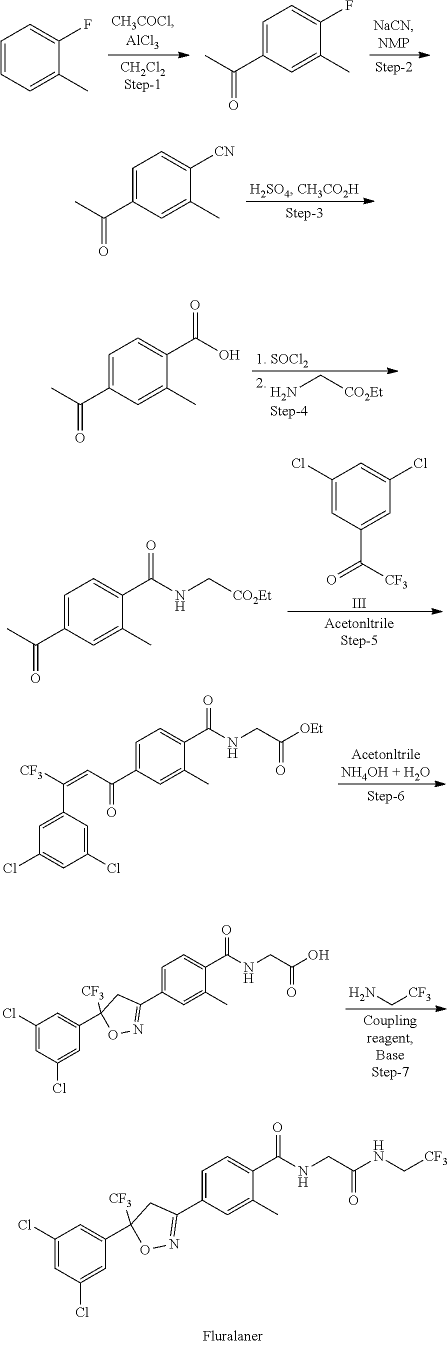

Step-1:1-(4-fluoro-3-methylphenyl)ethan-1-one

To a solution of 1-fluoro-2-methylbenzene (50 g, 454.0 mmol) in dichloromethane (250 ml) was added acetyl chloride (35.64 g, 454.0 mmol) at 25° C. followed by aluminum trichloride (AlCl₃) portion wise (72.64 g, 544.8 mmol) and stirred at 25° C. for 4 hours. After completion of the reaction, the reaction was quenched by pouring the reaction mass onto diluted HCl in ice water. The reaction mixture was extracted with dichloromethane (3×200 mL) and combined organic phase was washed with water (3×250 mL), dried over anhydrous sodium sulphate (Na₂SO₄). Dichloromethane was evaporated under reduced pressure to afford crude product 65 g, which was further purified by column chromatography to obtain 1-(4-fluoro-3-methylphenyl)ethan-1-one (40 g, 57%).

¹H NMR (300 MHZ, CDCl₃): δ 7.54-7.83 (m, 2H), 7.05 (t, J=9, 1H), 3.12 (s, 3H), 2.32 (s, 3H).

LC-MS (m/z): 152.1 (M+)

Step-2:4-acetyl-2-methylbenzonitrile

To a solution of 1-(4-fluoro-3-methylphenyl) ethan-1-one (40 g, 262.87 mmol) in NMP (200 ml) was added sodium cyanide (15.45 g, 315.44 mmol) at 25° C. and stirred at 130° C. for 15 hours. After completion of the reaction, the reaction mass was cooled to 25° C., diluted by dichloromethane and washed with water. The dichloromethane was separated and dried over anhydrous sodium sulphate. Dichloromethane was evaporated under reduced pressure to afford crude compound (35 g) which was further purified by column chromatography to obtain the 4-acetyl-2-methylbenzonitrile (27 g, 64%).

¹H NMR (300 MHZ, CDCl₃): δ 7.87-7.88 (m, 1H), 7.81-7.84 (m, 1H), 7.70 (d, J=8.1 Hz, 1H), 2.65 (s, 6H).

LC-MS (m/z): 318.76 (2M+H)

Step-3:4-acetyl-2-methylbenzoic acid

To a solution of 4-acetyl-2-methylbenzonitrile (27 g, 169.6 mmol) in water (150 mL) was added sulphuric acid (10 G) and glacial acetic acid (100 mL) at 25° C. and stirred at 100° C. 12 hours. After completion of the reaction, the reaction mass was cooled to 25° C., diluted by adding cold water, and basified (to pH 10.0) by sodium hydroxide (30%). The aqueous layer was washed with ethyl acetate followed by acidification by 6N hydrogen chloride, and by ethyl acetate. The ethyl acetate layer was separated and dried over anhydrous sodium sulphate (Na₂SO₄) to obtain 4-acetyl-2-methylbenzoic acid (23 g, 76%).

¹H NMR (300 MHz, CDCl₃): δ 8.12 (d, J=8.1 Hz, 1H), 7.81-7.84 (m, 2H), 2.71 (s, 3H), 2.64 (s, 3H).

LC-MS (m/z): 178.2 (M)

Step-4: ethyl (4-acetyl-2-methylbenzoyl)glycinate

To a solution of 4-acetyl-2-methylbenzoic acid (1.9 g, 10.66 mmol) in dichloromethane (10 mL) was added thionyl chloride (1.25 g, 10.66 mmol) at 25° C. and stirred at 40° C. for 5 hours. After completion of the reaction, the reaction mass was cooled to 25° C. and added to the solution of ethyl glycinate hydrochloride (1.64 g, 11.73 mmol) in dichloromethane (10 mL) and triethyl amine (3.5 mL, 42.65 mmol) at 0° C. and stirred at 25° C. for 2 hours. After completion of the reaction, the reaction mass was diluted with dichloromethane (10 mL) and washed with water (2×15 mL). The dichloromethane layer was separated, dried over anhydrous sodium sulphate (Na₂SO₄) and concentrated under vacuum to obtain ethyl (4-acetyl-2-methylbenzoyl) glycinate (2.7 g, 96%).

¹H NMR (300 MHZ, CDCl₃): δ 7.44-7.78 (m, 2H), 7.48 (d, J=7.8 Hz, 1H), 6.41 (s, 1H), 4.18-4.28 (m, 2H), 2.58 (s, 3H), 2.49 (s, 3H), 1.30 (t, J=7.2 Hz, 3H)

LC-MS (m/z): 264.10 (M+H)⁺

Step-5: ethyl (E)-(4-(3-(3,5-dichlorophenyl)-4,4,4-trifluorobut-2-enoyl)-2-methylbenzoyl) glycinate

To a solution of ethyl (4-acetyl-2-methylbenzoyl) glycinate (1.3 g, 4.94 mmol) in acetonitrile (20 mL) was added cesium carbonate (6.43 g, 19.75 mmol) at 25° C. followed by 1-(3,5-dichlorophenyl)-2,2,2-trifluoroethan-1-one (1.44 g, 5.92 mmol) and refluxed. After completion of the reaction, the reaction mass was cooled to 25° C., filtered to remove cesium carbonate and solvent was distilled out. Water (20 mL) was added to the reaction mixture and extracted with ethyl acetate (x15 mL). The ethyl acetate layer was washed with water, dried over anhydrous sodium sulphate (Na₂SO₄) and distilled out under reduced pressure to obtain ethyl (E)-(4-(3-(3,5-dichlorophenyl)-4,4,4-trifluorobut-2-enoyl)-2-methylbenzoyl) glycinate (1.97 g, yield 82%).

¹H NMR (300 MHZ, CDCl₃): δ 7.52 (d, J-8.7 Hz, 2H), 7.29-7.34 (m, 3H), 7.23 (t, J=1.8 Hz, 1H), 7.065 (d, J=1.5 Hz, 2H), 6.57 (s, 1H) 4.13 (q, J=6.9 Hz, 2H), 4.05 (d, J=5.4 Hz, 2H), 2.34 (s, 3H), 1.30 (t, J=7.2 Hz, 3H).

LC-MS (m/z): 487.97 (M+H)⁺

Step-6: ethyl (4-(5-(3,5-dichlorophenyl)-5-(trifluoromethyl)-4,5-dihydroisoxazol-3-yl)-2-methylbenzoyl) glycinate

To a solution of ethyl (E)-(4-(3-(3,5-dichlorophenyl)-4,4,4-trifluorobut-2-enoyl)-2-methylbenzoyl) glycinate (1 g, 2.05 mmol) in acetonitrile (10 mL) was added DBU (623.5 mg, 4.10 mmol), hydroxylamine 50% in water (0.6 mL, 8.19 mmol), and tert-butyl ammonium bromide (50 mg) and lithium hydroxide (133.4 mg, dissolved in 1 mL water) and stirred at 25° C. for 2 hours. After completions of the reaction, ethanol was evaporated, diluted with water and acidified by conc. Hydrochloric acid to precipitate the product which was then filtered, washed with water and traces of water were evaporated under reduced pressure to obtain 4-(5-(3,5-dichlorophenyl)-5-(trifluoromethyl)-4,5-dihydroisoxazol-3-yl)-2-methylbenzoyl) glycine (850 mg, yield 87%).

¹H NMR (300 MHz, CDCl₃): δ 7.40-7.49 (m, 6H), 6.73 (s, 1H), 4.19 (s, 2H), 4.07 (d, J=17.4 Hz, 1H), 3.70 (d, J=17.4 Hz, 1H), 2.39 (s, 3H).

LC-MS (m/z): 475 (M+H)⁺

Step-7:4-(5-(3,5-dichlorophenyl)-5-(trifluoromethyl)-4,5-dihydroisoxazol-3-yl)-2-methyl-N-(2-oxo-2-((2,2,2-trifluoroethyl) amino) ethyl) benzamide (Fluralaner-Compound of Formula I)

To a solution of (4-(5-(3,5-dichlorophenyl)-5-(trifluoromethyl)-4,5-dihydroisoxazol-3-yl)-2-methylbenzoyl) glycine (800 mg, 1.68 mmol) in dichloromethane was added 2,2,2-trifluoroethan-1-amine hydrochloride (249.3 mg, 1.84 mmol) followed by addition of triethylamine (42.59 mg, 4.2 mmol) and EDC.HCl. (362.6 mg, 1.84 mmol) at 25° C. The reaction mass was stirred at 25° C. After completion of the reaction, the reaction mass was diluted with water and extracted by dichloromethane. The separated dichloromethane layer was dried over anhydrous sodium sulphate (Na₂SO₄) and concentrated under reduced pressure to obtain 4-(5-(3,5-dichlorophenyl)-5-(trifluoromethyl)-4,5-dihydroisoxazol-3-yl)-2-methyl-N-(2-oxo-2-((2,2,2-trifluoroethyl) amino) ethyl) benzamide (Fluralaner-Compound of Formula I) 110 mg (769 mg, yield: 82%).

¹H NMR (300 MHz, CDCl₃): δ 7.58-7.47 (m, 6H), 6.97-6.95 (m, 1H), 6.80-6.77 (m, 1H), 4.26-4.25 (d, 2H), 4.16-4.12 (d, 1H), 4.01-3.92 (m, 2H), 3.73-3.69 (d, 1H), 2.47 (s, 3H).

LC-MS (m/z): 556.15 & 558.15 (M+H)⁺

### Example 2: Preparation of Fluralaner

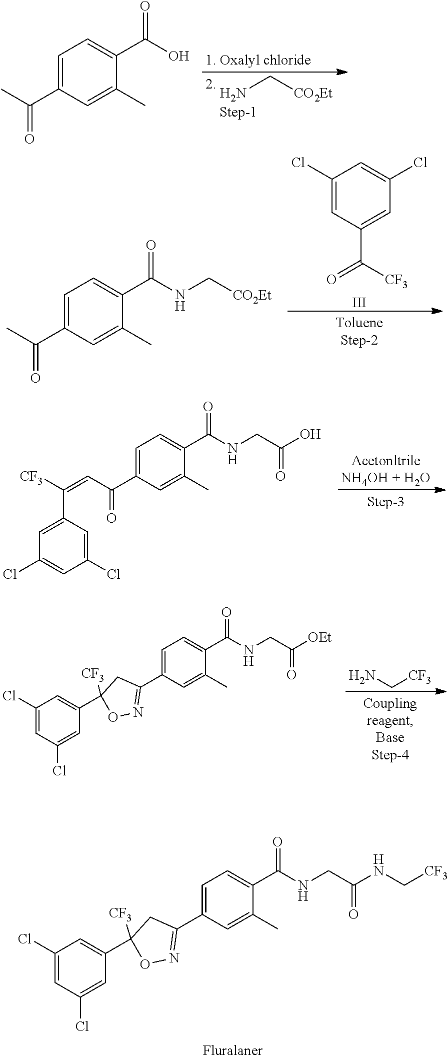

Step-1: (4-acetyl-2-methylbenzoyl)glycine

To a solution of 4-acetyl-2-methylbenzoic acid (50 g, 27.97 mmol) in dichloromethane (150 mL) was added oxalyl chloride (53.4 g, 42.07 mmol) and 0.3 mL of dimethylformamide at 25° C. and stirred 5 hours. After completion of the reaction, the volatiles were distilled off under reduced pressure. The residue was dissolved in tetrahydrofuran (150 mL) and the resulting solution was added to the solution of glycine hydrochloride (21.0 g, 27.97 mmol) in aqueous (42 mL) sodium hydroxide (22.37 g, 55.94 mmol) at 0° C. and stirred at 0° C. for 8 hours. After completion of the reaction, toluene (165 mL) was added to the reaction mixture. Layers were separated. pH of the aqueous layer was adjusted to 1 with 12 M aqueous hydrochloric acid. Precipitate was filtered and washed with water (165 mL) to obtain ethyl (4-acetyl-2-methylbenzoyl) glycinate (62 g, 94%).

¹H NMR (300 MHz, CDCl₃): δ 8.74-8.71 (m, 1H), 7.84-7.82 (dm 2H), 7.84-7.82 (d, 1H), 3.92-3.91 (d, 2H), 2.60 (s, 3H), 2.43 (s, 3H) LC-MS (m/z): 234.10 (M−H)+

Step-2: (E)-(4-(3-(3,5-dichlorophenyl)-4,4,4-trifluorobut-2-enoyl)-2-methylbenzoyl) glycine

To a solution of (4-acetyl-2-methylbenzoyl) glycine (50 g, 21.3 mmol) in toluene (150 mL) was added 1,8-Diazabicyclo (5.4.0) undec-7-ene (32.4 g, 21.3 mmol) at 25° C. followed by 1-(3,5-dichlorophenyl)-2,2,2-trifluorocthan-1-one (56.9 g, 23 mmol) and heated at 110° C. for 6 hours. After completion of the reaction, the volatiles were distilled out under reduced pressure and the residue was triturated with 1 M aqueous hydrochloric acid (150 ml). The precipitate formed was filtered and washed with water (100 mL) and air dried to obtain (E)-(4-(3-(3,5-dichlorophenyl)-4,4,4-trifluorobut-2-enoyl)-2-methylbenzoyl) glycine (83 g, yield 85%)

¹H NMR (300 MHz, DMSO-d6): δ 12.62 (s, 1H), 8.77-8.76 (m, 1H), 7.92 (s, 1H), 7.83-7.38 (m, 5H), 3.94-3.90 (m, 2H) 2.51 (s, 3H), 2.46 (s, 3H).

LC-MS (m/z): 460.07 & 462.07 (M+H)⁺

Step-3: (4-(5-(3,5-dichlorophenyl)-5-(trifluoromethyl)-4,5-dihydroisoxazol-3-yl)-2-methylbenzoyl) glycine

To a solution of (E)-(4-(3-(3,5-dichlorophenyl)-4,4,4-trifluorobut-2-enoyl)-2-methylbenzoyl) glycine (50 g, 10.86 mmol) in dioxane (100 mL) was added hydroxylamine hydrochloride (15.1 g, 21.72 mmol) and 8.5 molar aqueous at 0° C. Stirred at 0° C. for 2 hours. Distill out the dioxane once the reaction conversion is completed. Water (150 mL) was added to the residue and pH was adjusted to 2.0. Filter the precipitate and wash with water 9100 mL). Air dried to obtain (4-(5-(3,5-dichlorophenyl)-5-(trifluoromethyl)-4,5-dihydroisoxazol-3-yl)-2-methylbenzoyl) glycine (47.5 g, yield 92%).

¹H NMR (300 MHZ, DMSO-d₆): δ 12.65 (s, 1H), 8.71-8.68 (t, 1H), 7.82 (m, 1H), 7.61 (m, 4H), 7.47-7.40 (m, 1H), 2.52 (s, 3H), 2.41 (s, 3H).

LC-MS (m/z): 475.15 & 477.15 (M+H)⁺

Step-4:4-(5-(3,5-dichlorophenyl)-5-(trifluoromethyl)-4,5-dihydroisoxazol-3-yl)-2-methyl-N-(2-oxo-2-((2,2,2-trifluoroethyl) amino) ethyl) benzamide (Fluralaner-Compound of Formula I)

To a solution of (4-(5-(3,5-dichlorophenyl)-5-(trifluoromethyl)-4,5-dihydroisoxazol-3-yl)-2-methylbenzoyl) glycine (20 g, 42.0 mmol) in tetrahydrofuran (60 mL) was added 2,2,2-trifluoroethan-1-amine hydrochloride (6.25 g, 46.12 mmol) followed by addition of triethylamine (10.2 g, 100.8 mmol), and EDC.HCl. (9.66 g, 50.4 mmol) at 25° C. The reaction mass was stirred at 25° C. for 12 h. After completion of the reaction, THF was distilled under reduced pressure. The residue was agitated with 1 M HCl (100 mL) at 0° C. The precipitate was filtered and washed with water (50 mL). Dried to obtain crude product, which was recrystallized from ethyl acetate and hexane to obtain 4-(5-(3,5-dichlorophenyl)-5-(trifluoromethyl)-4,5-dihydroisoxazol-3-yl)-2-methyl-N-(2-oxo-2-((2,2,2-trifluoroethyl) amino) ethyl) benzamide (Fluralaner-Compound of Formula I) (19.4 g, yield 83%).

¹H NMR (300 MHz, CDCl₃): δ 7.56-7.45 (m, 6H), 6.99-6.96 (m, 1H), 6.76-6.73 (m, 1H), 4.24-4.23 (d, 2H), 4.13-4.09 (d, 1H), 4.02-3.93 (m, 2H), 3.75-3.71 (d, 1H), 2.49 (s, 3H).

LC-MS (m/z): 556.15 & 558.15 (M+H)⁺

### Example 3: Preparation of Fluralaner

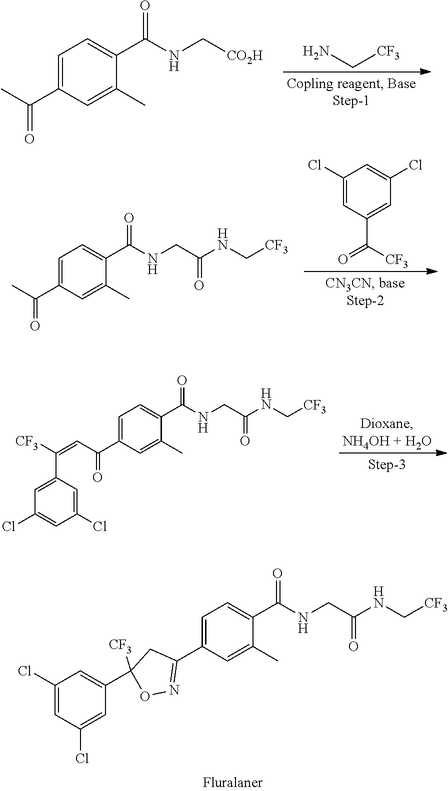 [SMILES: CC(=O)c1ccc(C(=O)NCC(=O)O)c(C)c1 ; NCC(F)(F)F ; CC(=O)c1ccc(C(=O)NCC(=O)NCC(F)(F)F)c(C)c1 ; O=C(c1cc(Cl)cc(Cl)c1)C(F)(F)F ; Cc1cc(C(=O)/C=C(\c2cc(Cl)cc(Cl)c2)C(F)(F)F)ccc1C(=O)NCC(=O)NCC(F)(F)F ; Cc1cc(C2=NOC(c3cc(Cl)cc(Cl)c3)(C(F)(F)F)C2)ccc1C(=O)NCC(=O)NCC(F)(F)F]

Step-1:4-acetyl-2-methyl-N-(2-oxo-2-((2,2,2-trifluoroethyl) amino) ethyl) benzamide

To a solution of 4-acetyl-2-methylbenzoyl) glycine (10 g, 42.5 mmol) in tetrahydrofuran (30 mL) was added 2,2,2-trifluoroethan-1-amine hydrochloride (6.25 g, 46.12 mmol) followed by addition of triethylamine (10.2 g, 100.8 mmol), and EDC.HCl. (9.66 g, 50.4 mmol) at 25° C. The reaction mass was stirred at 25° C. for 12 h. After completion of the reaction, THF was distilled under reduced pressure. The residue was agitated with 1 M HCl (100 mL) at 0° C. The precipitate was filtered and washed with water (50 mL). Dried to obtain crude product, which was recrystallized from ethyl acetate and hexane to obtain4-(5-(3,5-dichlorophenyl)-5-(trifluoromethyl)-4,5-dihydroisoxazol-3-yl)-2-methyl-N-(2-oxo-2-((2,2,2-trifluoroethyl) amino) ethyl) benzamide (Fluralaner-Compound of Formula I) (12.1 g, yield 90%).

LC-MS (m/z): 317.12 (M−H)⁺

Step-2: (E/Z)-(4-(3-(3,5-dichlorophenyl)-4,4,4-trifluorobut-2-enoyl)-2-methylbenzoyl) glycine

To a solution of 4-acetyl-2-methyl-N-(2-oxo-2-((2,2,2-trifluoroethyl) amino) ethyl) benzamide (10 g, 31.6 mmol) in toluene (30 mL) was added 1,8-Diazabicyclo (5.4.0) undec-7-ene (4.8 g, 31.6 mmol) at 25° C. followed by 1-(3,5-dichlorophenyl)-2,2,2-trifluoroethan-1-one (7.7 g, 31.6 mmol) and heated at 110° C. for 6 hours. After completion of the reaction, the volatiles were distilled out under reduced pressure. The residue was dissolved in ethyl acetate (100 mL) and washed with 1.0 M aqueous hydrochloric acid and water (100 mL). the organic phase was dried over sodium sulphate and evaporated to obtain (E)-(4-(3-(3,5-dichlorophenyl)-4,4,4-trifluorobut-2-enoyl)-2-methylbenzoyl) glycine (12.8 g, yield 75%) LC-MS (m/z): 540.07 & 542.08 (M+H)⁺

Step-4:4-(5-(3,5-dichlorophenyl)-5-(trifluoromethyl)-4,5-dihydroisoxazol-3-yl)-2-methyl-N-(2-oxo-2-((2,2,2-trifluoroethyl) amino) ethyl) benzamide (Fluralaner-Compound of Formula I)

To a solution of (E/Z)-(4-(3-(3,5-dichlorophenyl)-4,4,4-trifluorobut-2-enoyl)-2-methylbenzoyl) glycine (10 g, 18.5 mmol) in tetrahydrofuran (30 mL) was added 2,2,2-trifluoroethan-1-amine hydrochloride (2.76 g, 20.3 mmol) followed by addition of triethylamine (4.5 g, 44.4 mmol), and EDC.HCl. (4.25 g, 22.2 mmol) at 25° C. The reaction mass was stirred at 25° C. for 12 h. After completion of the reaction, THF was distilled under reduced pressure. The residue was agitated with 1 M HCl (100 mL) at 0° C. The precipitate was filtered and washed with water (50 mL). Dried to obtain crude product, which was recrystallized from ethyl acetate and hexane to obtain 4-(5-(3,5-dichlorophenyl)-5-(trifluoromethyl)-4,5-dihydroisoxazol-3-yl)-2-methyl-N-(2-oxo-2-((2,2,2-trifluoroethyl) amino) ethyl) benzamide (Fluralaner-Compound of Formula I) (8.45 g, yield 82%).

¹H NMR (300 MHz, CDCl₃): δ 7.40-7.49 (m, 6H), 6.95-6.93 (m, 1H), 6.73-6.70 (m, 1H), 4.29-4.28 (d, 2H), 4.07-4.03 (d, 1H), 4.04-3.95 (m, 2H), 3.70-3.63 (d, 1H), 2.42 (s, 3H).

LC-MS (m/z): 556.15 & 558.15 (M+H)⁺

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The use of the expression “at least” or “at least one” suggests the use of one or more elements or ingredients or quantities, as the use may be in the embodiment of the disclosure to achieve one or more of the desired objects or results.

The numerical values mentioned for the various physical parameters, dimensions or quantities are only approximations and it is envisaged that the values higher/lower than the numerical values assigned to the parameters, dimensions or quantities fall within the scope of the disclosure, unless there is a statement in the specification specific to the contrary.

While considerable emphasis has been placed herein on the particular features of this disclosure, it will be appreciated that various modifications can be made, and that many changes can be made in the preferred embodiments without departing from the principles of the disclosure. These and other modifications in the nature of the disclosure or the preferred embodiments will be apparent to those skilled in the art from the disclosure herein, whereby it is to be distinctly understood that the foregoing descriptive matter is to be interpreted merely as illustrative of the disclosure and not as a limitation.

1. A process for the preparation of a compound of Formula I, 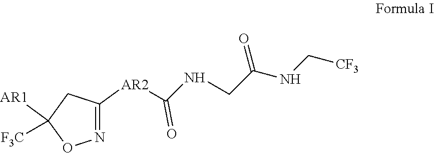
wherein,
AR1 is 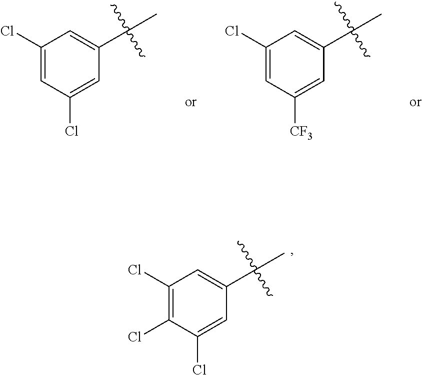 [SMILES: *c1cc(Cl)cc(Cl)c1 ; *c1cc(Cl)cc(C(F)(F)F)c1 ; *c1cc(Cl)c(Cl)c(Cl)c1]
and
AR2 is 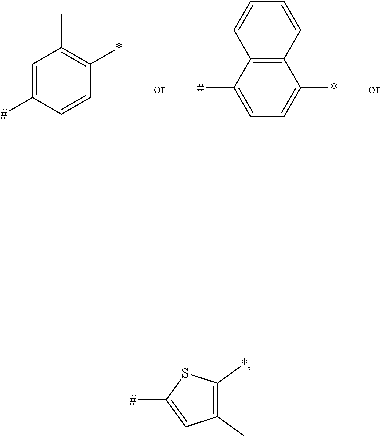
#indicates the attachment to isoxazoline ring and * indicates the attachment to the carbonyl carbon,
said process comprising the following steps:
a) reacting a compound of Formula II 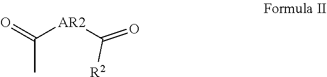
wherein,
AR2 is 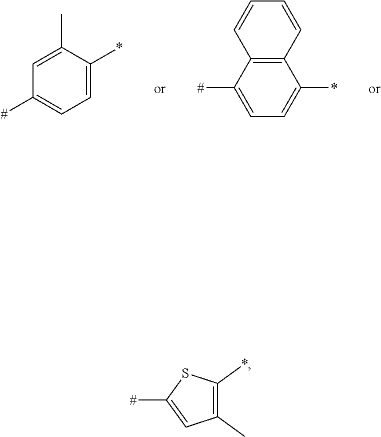
#indicates the attachment to isoxazoline ring and * indicates the attachment to the carbonyl carbon, and
R² is OH, F, Cl or OR³ & R³ is straight or branched chain C₁-C₄ alkyl, with a compound of Formula III 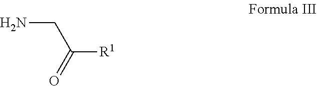
wherein, R¹ is selected from the group consisting of —OH, —OMe, —OEt, —O(n)Pr, —O(i)Pr, —O(n)Bu, —O(i)Bu, —O(sec)Bu, and —O(tert)Bu, to obtain a compound of Formula IV; 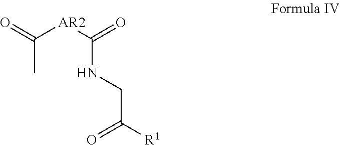
wherein,
AR2 is 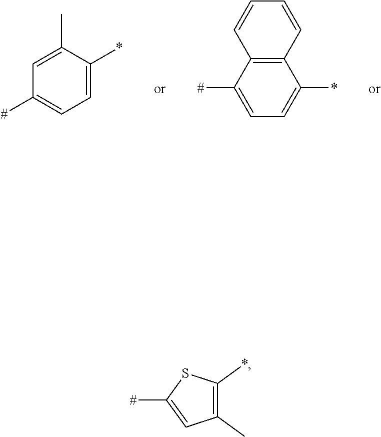
#indicates the attachment to isoxazoline ring and * indicates the attachment to the carbonyl carbon,
R¹ is selected from the group consisting of —OH, —OMe, —OEt, —O(n)Pr, —O(i)Pr, —O(n)Bu, —O(i)Bu, —O(sec)Bu, and —O(tert)Bu,
b) reacting the compound of Formula IV with a compound of Formula V 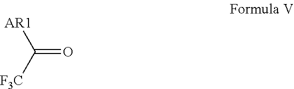
wherein,
AR1 is 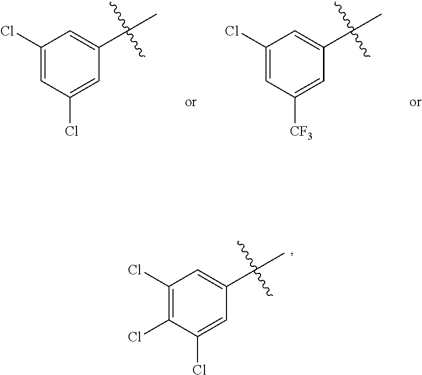 [SMILES: *c1cc(Cl)cc(Cl)c1 ; *c1cc(Cl)cc(C(F)(F)F)c1 ; *c1cc(Cl)c(Cl)c(Cl)c1]
to obtain a compound of Formula VI(i) which is converted to a compound of Formula VI(ii); 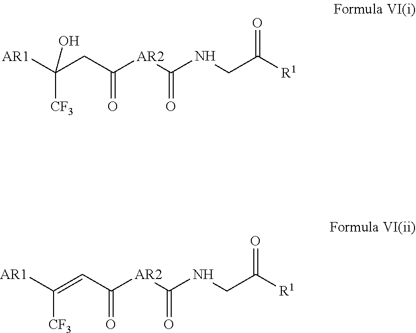
wherein,
AR1 is 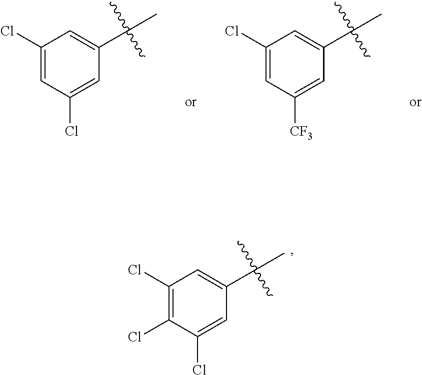 [SMILES: *c1cc(Cl)cc(Cl)c1 ; *c1cc(Cl)cc(C(F)(F)F)c1 ; *c1cc(Cl)c(Cl)c(Cl)c1]
AR2 is 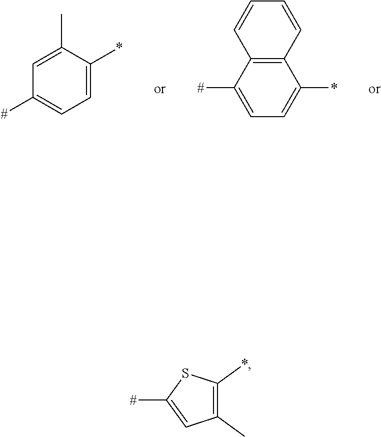
#indicates the attachment to isoxazoline ring and * indicates the attachment to the carbonyl carbon, and
R¹ is selected from the group consisting of —OH, —OMe, —OEt, —O(n)Pr, —O(i)Pr, —O(n)Bu, —O(i)Bu, —O(sec)Bu, and —O(tert)Bu,
c) converting the compound of Formula VI(ii) into a compound of Formula VII; 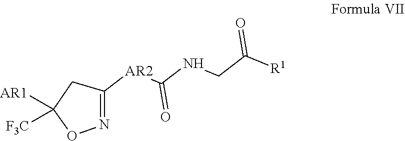
wherein,
AR1 is 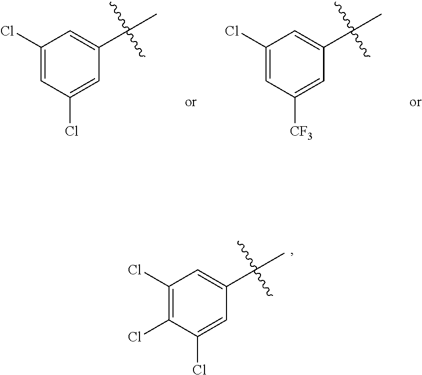 [SMILES: *c1cc(Cl)cc(Cl)c1 ; *c1cc(Cl)cc(C(F)(F)F)c1 ; *c1cc(Cl)c(Cl)c(Cl)c1]
AR2 is 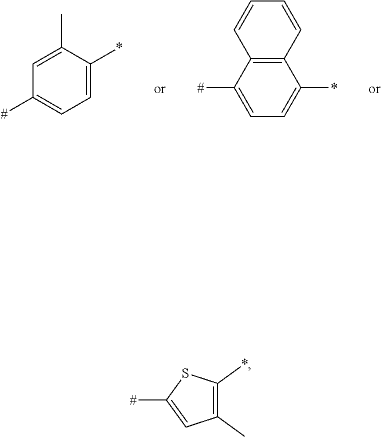
#indicates the attachment to isoxazoline ring and * indicates the attachment to the carbonyl carbon, and
R¹ is selected from the group consisting of —OH, —OMe, —OEt, —O(n)Pr, —O(i)Pr, —O(n)Bu, —O(i)Bu, —O(sec)Bu, and —O(tert)Bu,
d) converting the compound of Formula VII into a compound of Formula VIII(i); 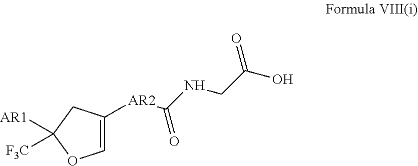
wherein,
AR1 is 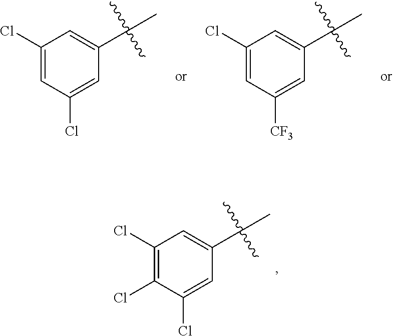 [SMILES: *c1cc(Cl)cc(Cl)c1 ; *c1cc(Cl)cc(C(F)(F)F)c1 ; *c1cc(Cl)c(Cl)c(Cl)c1]
and
AR2 is 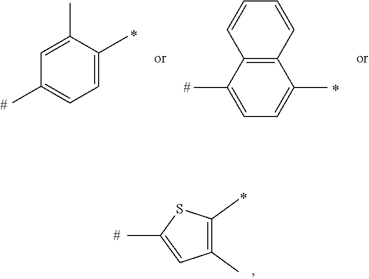
#indicates the attachment to isoxazoline ring and * indicates the attachment to the carbonyl carbon,
e) optionally, converting the compound of Formula VIII(i) into a compound of Formula VIII(ii); and 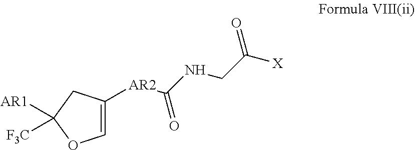
wherein,
AR1 is 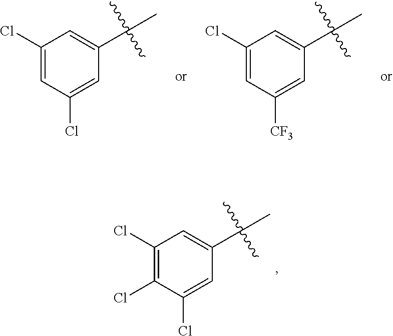 [SMILES: *c1cc(Cl)cc(Cl)c1 ; *c1cc(Cl)cc(C(F)(F)F)c1 ; *c1cc(Cl)c(Cl)c(Cl)c1]
AR2 is 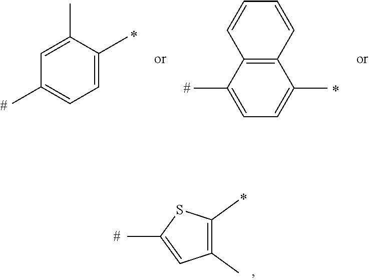
#indicates the attachment to isoxazoline ring and * indicates the attachment to the carbonyl carbon, and
X is Cl or F,
f) reacting the compound of Formula VII or VIII(i) or VIII(ii) with a compound of Formula IX 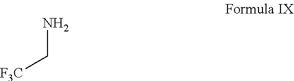
to obtain the compound of Formula I.

2. A process for preparing a compound of Formula I, said process comprising the following steps:
a) reacting the compound of Formula IV 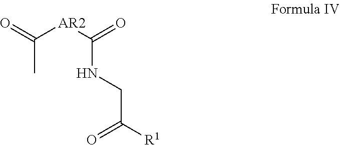
AR2 is 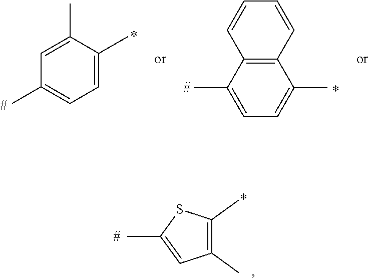
#indicates the attachment to isoxazoline ring and * indicates the attachment to the carbonyl carbon,
R¹ is selected from the group consisting of —OH, —OMe, —OEt, —O(n)Pr, —O(i)Pr, —O(n)Bu, —O(i)Bu, —O(sec)Bu, and —O(tert)Bu,
with the compound of Formula IX 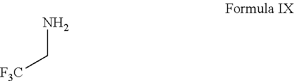
to obtain a compound of Formula X, 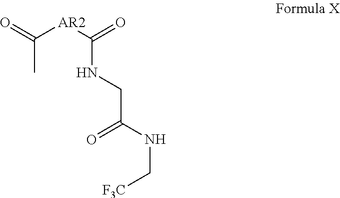
b) reacting the compound of Formula X with the compound of Formula V 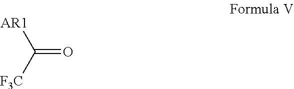
wherein,
AR1 is 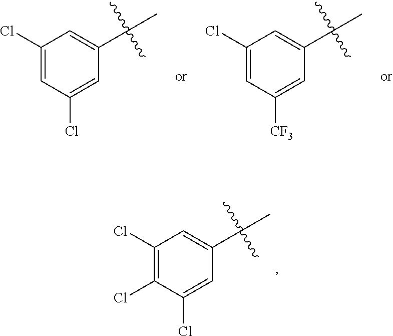 [SMILES: *c1cc(Cl)cc(Cl)c1 ; *c1cc(Cl)cc(C(F)(F)F)c1 ; *c1cc(Cl)c(Cl)c(Cl)c1]
to obtain a compound of Formula XI(i) which is converted to a compound of Formula XI(ii); 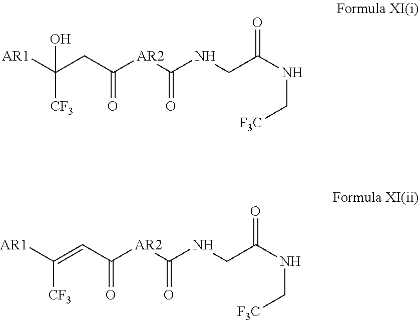
wherein,
AR1 is 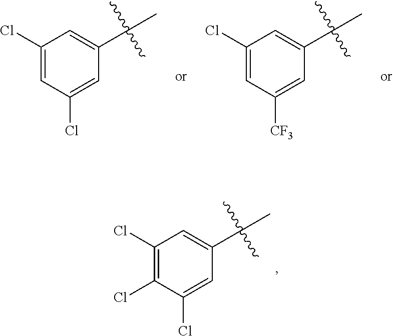 [SMILES: *c1cc(Cl)cc(Cl)c1 ; *c1cc(Cl)cc(C(F)(F)F)c1 ; *c1cc(Cl)c(Cl)c(Cl)c1]
AR2 is 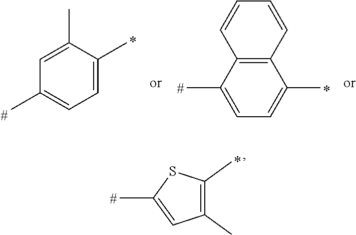
and
#indicates the attachment to isoxazoline ring and * indicates the attachment to the carbonyl carbon,
c) converting the compound of Formula XI(ii) into the compound of Formula I; 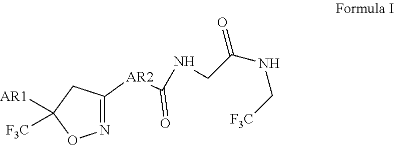
wherein,
AR1 is 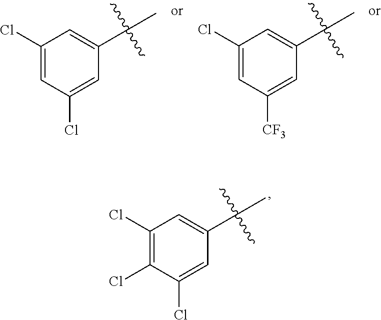 [SMILES: *c1cc(Cl)cc(Cl)c1 ; *c1cc(Cl)cc(C(F)(F)F)c1 ; *c1cc(Cl)c(Cl)c(Cl)c1]
AR2 is 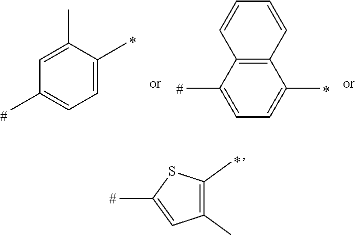
and
#indicates the attachment to isoxazoline ring and * indicates the attachment to the carbonyl carbon.

3. The process as claimed in claim 1, wherein the compound of Formula II is prepared from a compound of Formula II-1, the process comprises the following steps:
acylating the compound of Formula II-1 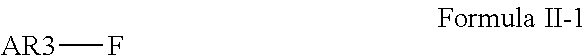
wherein, AR3 is 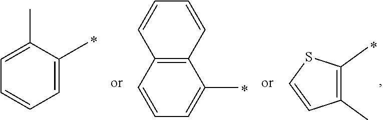
* indicates the point of attachment to F atom
to obtain a compound of Formula II-2, 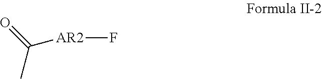
wherein, AR2 is 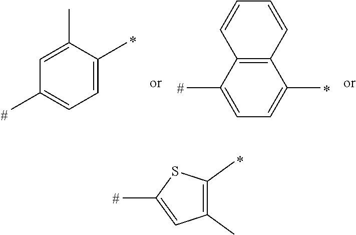
#indicates the attachment to the carbonyl carbon and * indicates the attachment to F atom,
converting the compound of Formula II-2 into a compound of Formula II-3 using a cyano reagent, and 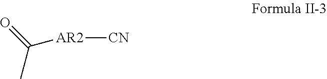
wherein, AR2 is 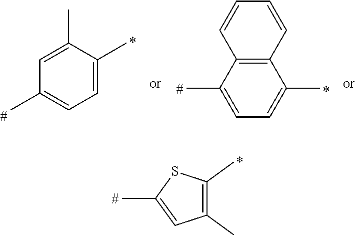
#indicates the attachment to the carbonyl carbon and * indicates the attachment to cyano group,
hydrolyzing the compound of Formula II-3 and optionally, chlorinating using a chlorinating agent to obtain the compound of Formula II. 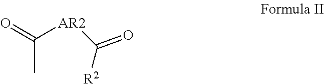
wherein,
AR2 is 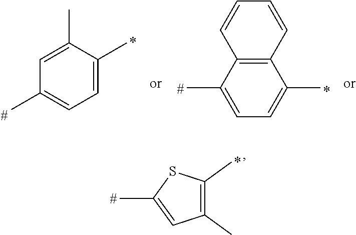
#indicates the attachment to isoxazoline ring and * indicates the attachment to the carbonyl carbon, and
R² is OH, F, Cl or OR³ & R³ is straight or branched chain C₁-C₄ alkyl.

4. A compound of Formula IV; 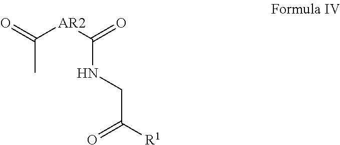
wherein
AR2 is 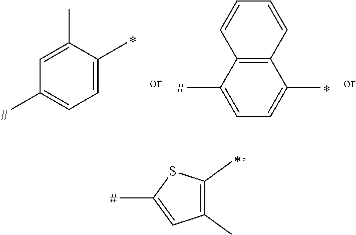
#indicates the attachment to isoxazoline ring and * indicates the attachment to the carbonyl carbon, and
R¹ is selected from the group consisting of —OH, —OMe, —OEt, —O(n)Pr, —O(i)Pr, —O(n)Bu, —O(i)Bu, —O(sec)Bu, and —O(tert)Bu.

5. A compound of Formula VI(i) and VI(ii); 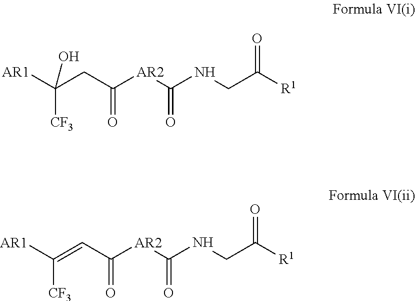
wherein,
AR1 is 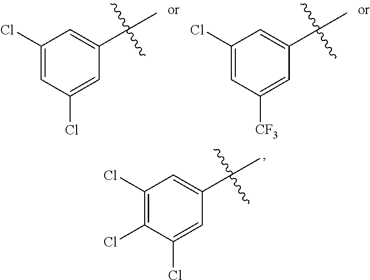 [SMILES: *c1cc(Cl)cc(Cl)c1 ; *c1cc(Cl)cc(C(F)(F)F)c1 ; *c1cc(Cl)c(Cl)c(Cl)c1]
and
AR2 is 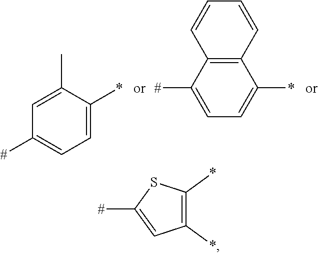
#indicates the attachment to isoxazoline ring and * indicates the attachment to the carbonyl carbon, and
R¹ is selected from the group consisting of —OH, —OMe, —OEt, —O(n)Pr, —O(i)Pr, —O(n)Bu, —O(i)Bu, —O(sec)Bu, and —O(tert)Bu.

6. A compound of Formula VII; 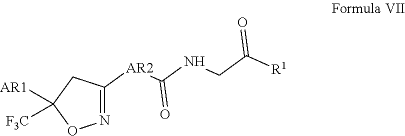
wherein
AR1 is 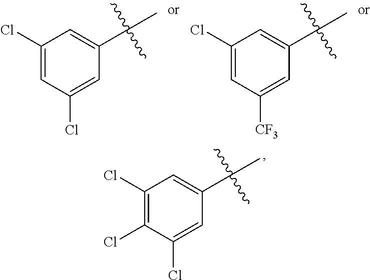 [SMILES: *c1cc(Cl)cc(Cl)c1 ; *c1cc(Cl)cc(C(F)(F)F)c1 ; *c1cc(Cl)c(Cl)c(Cl)c1]
and
AR2 is 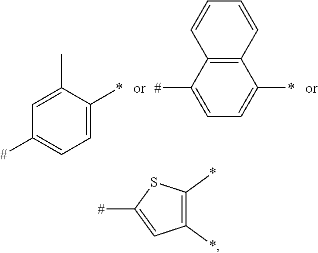
#indicates the attachment to isoxazoline ring and * indicates the attachment to the carbonyl carbon, and
R¹ is selected from the group consisting of —OH, —OMe, —OEt, —O(n)Pr, —O(i)Pr, —O(n)Bu, —O(i)Bu, —O(sec)Bu, and —O(tert)Bu.

7. A process for the preparation of compound of Formula I-a (Fluralaner), 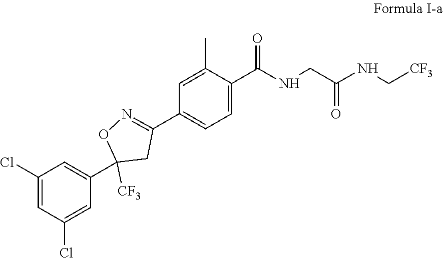 [SMILES: Cc1cc(C2=NOC(c3cc(Cl)cc(Cl)c3)(C(F)(F)F)C2)ccc1C(=O)NCC(=O)NCC(F)(F)F]
said process comprising the following steps:
a) reacting a compound of Formula II-a 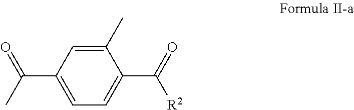
wherein, R² is OH, F, Cl or OR³ & R³ is straight or branched chain C₁-C₄ alkyl, with a compound of Formula III 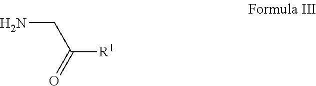
wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
to obtain a compound of Formula IV-a; 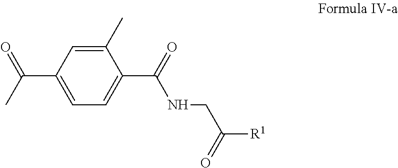
wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
b) reacting the compound of Formula IV-a with a compound of Formula V-a 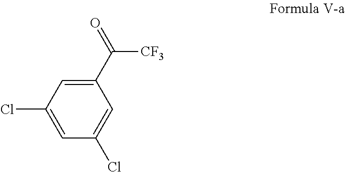 [SMILES: O=C(c1cc(Cl)cc(Cl)c1)C(F)(F)F]
to obtain a compound of Formula VI(i)-a which is converted to a compound of Formula VI(ii)-a; 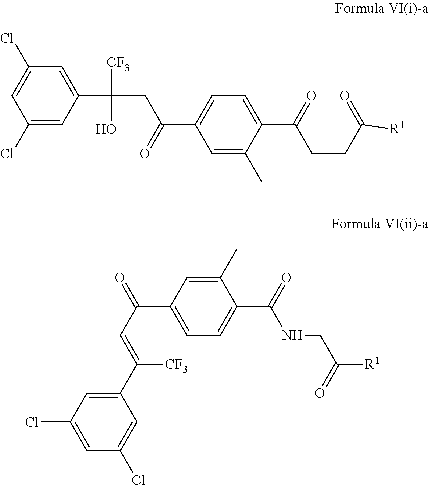
wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
c) converting the compound of Formula VI(ii)-a into a compound of Formula VII-a; 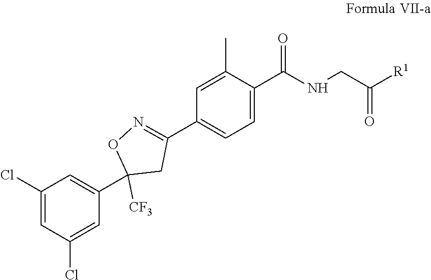
wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
d) converting the compound of Formula VII-a 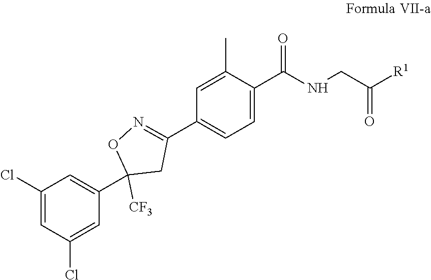
wherein, R¹ is selected from the group consisting of —OMe, —OEt, —O(n)Pr, —O(i)Pr, —O(n)Bu, —O(i)Bu, —O(sec)Bu, and —O(tert)Bu,
into a compound of Formula VIII(i)-a; 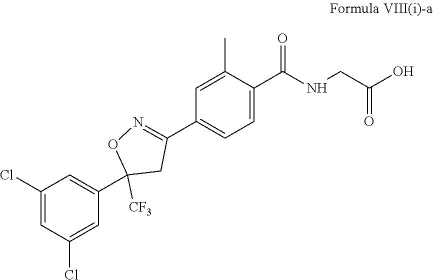 [SMILES: Cc1cc(C2=NOC(c3cc(Cl)cc(Cl)c3)(C(F)(F)F)C2)ccc1C(=O)NCC(=O)O]
e) optionally, converting the compound of Formula VIII(i)-a into a compound of Formula VIII(ii)-a; and 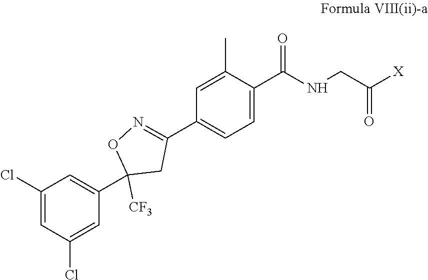
wherein, X is Cl or F,
f) reacting the compound of Formula VII-a or VIII(i)-a or VIII(ii)-a with the compound of Formula IX 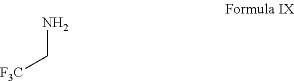
to obtain the compound of formula I-a.

8. A process for the preparation of compound of Formula I-a, the process comprising the steps of:
a) reacting the compound of Formula IV-a 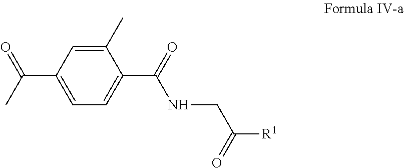
wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
with the compound of Formula IX 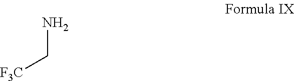
to obtain a compound of Formula X-a, 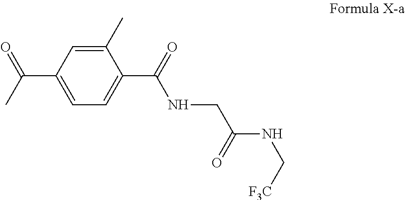 [SMILES: CC(=O)c1ccc(C(=O)NCC(=O)NCC(F)(F)F)c(C)c1]
b) reacting the compound of Formula X-a with the compound of Formula V-a 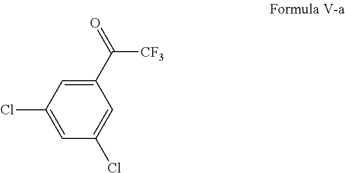 [SMILES: O=C(c1cc(Cl)cc(Cl)c1)C(F)(F)F]
to obtain a compound of Formula XI(i)-a which is converted to a compound of Formula XI(ii)-a; 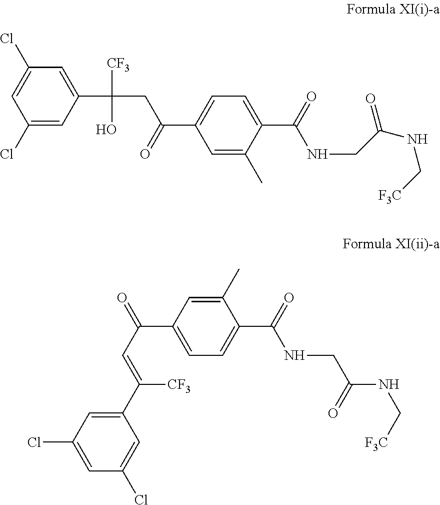 [SMILES: Cc1cc(C(=O)CC(O)(c2cc(Cl)cc(Cl)c2)C(F)(F)F)ccc1C(=O)NCC(=O)NCC(F)(F)F ; Cc1cc(C(=O)/C=C(/c2cc(Cl)cc(Cl)c2)C(F)(F)F)ccc1C(=O)NCC(=O)NCC(F)(F)F]
c) converting the compound of Formula XI(ii)-a into the compound of Formula I-a.

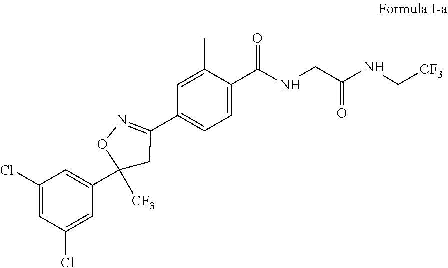 [SMILES: Cc1cc(C2=NOC(c3cc(Cl)cc(Cl)c3)(C(F)(F)F)C2)ccc1C(=O)NCC(=O)NCC(F)(F)F]

9. A process for the preparation of a compound of Formula II-a, said process comprising the following steps:
acylating the 2-fluro toluene to obtain a compound of Formula II-1-a. 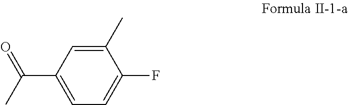
converting the compound of Formula II-1-a into a compound of Formula II-2-a using a cyano reagent. 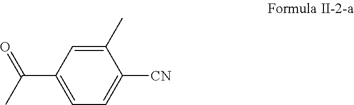
hydrolyzing the compound of Formula II-2-a and optionally, chlorinating using a chlorinating agent to obtain the compound of Formula II-a.

10. A compound of Formula IV-a; 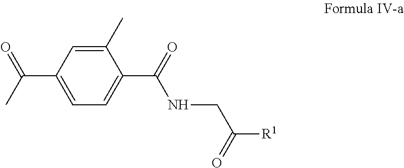
wherein, R¹ is selected from the group consisting of —OH, —OMe, —OEt, —O(n)Pr, —O(i)Pr, —O(n)Bu, —O(i)Bu, —O(sec)Bu, and —O(tert)Bu.

11. A compound of Formula VI(i)-a & VI(ii)-a; 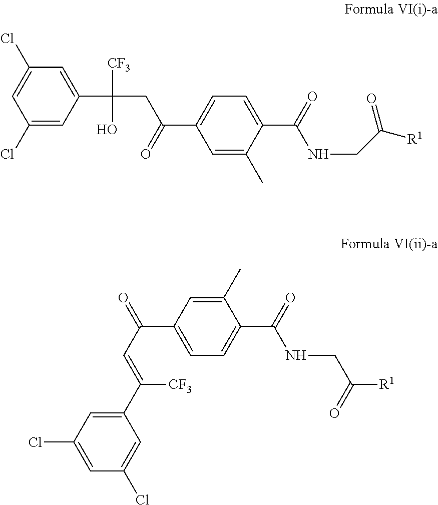
wherein, R¹ is selected from the group consisting of —OH, —OMe, —OEt, —O(n)Pr, —O(i)Pr, —O(n)Bu, —O(i)Bu, —O(sec)Bu, and —O(tert)Bu.

12. A compound of Formula VII-a; 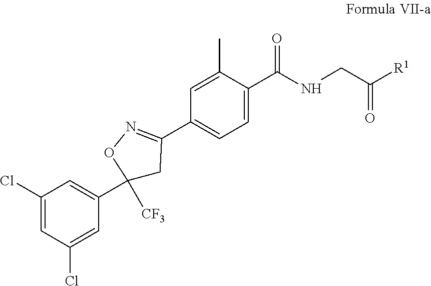
wherein, R¹ is selected from the group consisting of —OEt, —O(n)Pr, —O(i)Pr, —O(n)Bu, —O(i)Bu, —O(sec)Bu, and —O(tert)Bu.

13. A compound of Formula XI(i)-a

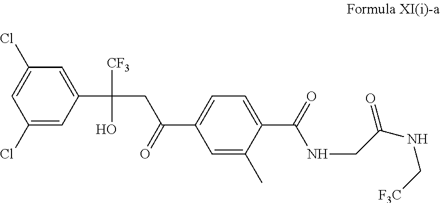 [SMILES: Cc1cc(C(=O)CC(O)(c2cc(Cl)cc(Cl)c2)C(F)(F)F)ccc1C(=O)NCC(=O)NCC(F)(F)F]

14. A process for the preparation of compound of Formula I-b (Afoxolaner), 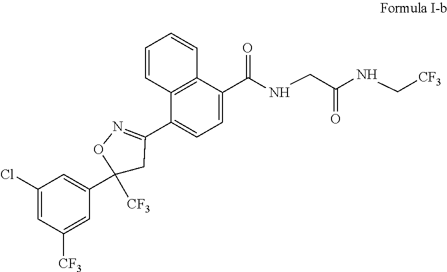 [SMILES: O=C(CNC(=O)c1ccc(C2=NOC(c3cc(Cl)cc(C(F)(F)F)c3)(C(F)(F)F)C2)c2ccccc12)NCC(F)(F)F]
said process comprising the following steps:
a) reacting a compound of Formula II-b 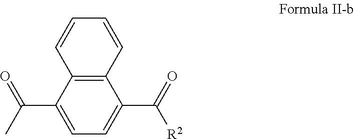
wherein, R² is OH, F, Cl or OR³ & R³ is straight or branched chain C₁-C₄ alkyl, with a compound of Formula III 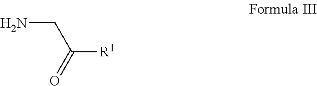
wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
to obtain a compound of Formula IV-b; 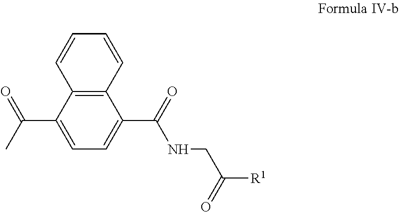
wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
b) reacting the compound of Formula IV-b with a compound of Formula V-b 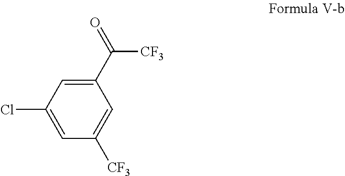 [SMILES: O=C(c1cc(Cl)cc(C(F)(F)F)c1)C(F)(F)F]
to obtain a compound of Formula VI(i)-b which is converted into a compound of Formula VI(ii)-b; 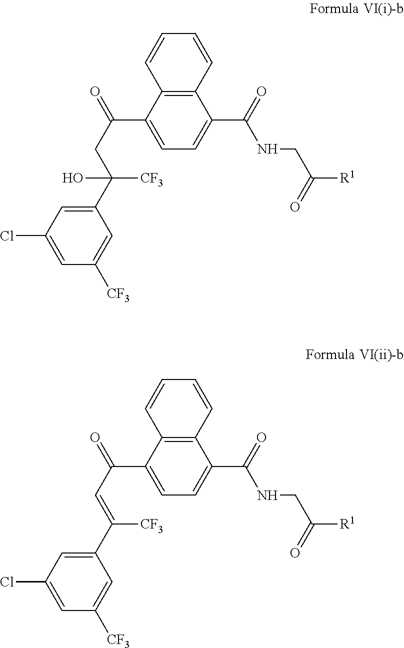
wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
c) converting the compound of Formula VI(ii)-b into a compound of Formula VII-b; 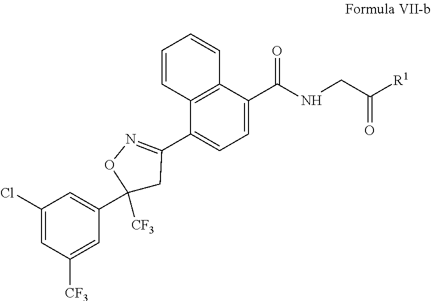
wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
d) converting the compound of Formula VII-b 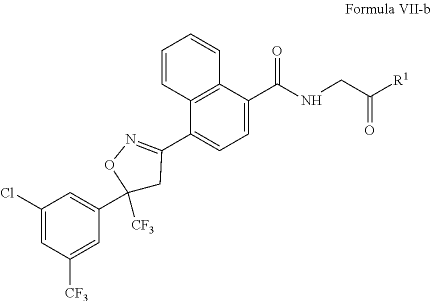
wherein, R¹ is selected from the group consisting of —OMe, —OEt, —O(n)Pr, —O(i)Pr, —O(n)Bu, —O(i)Bu, —O(sec)Bu, and —O(tert)Bu,
into a compound of Formula VIII(i)-b; 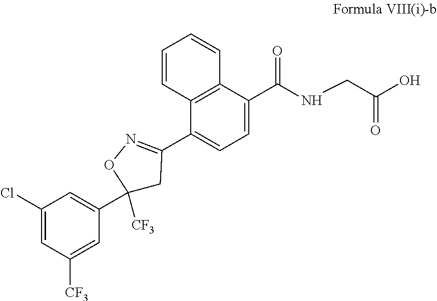 [SMILES: O=C(O)CNC(=O)c1ccc(C2=NOC(c3cc(Cl)cc(C(F)(F)F)c3)(C(F)(F)F)C2)c2ccccc12]
e) optionally, converting the compound of Formula VIII(i)-b into a compound of Formula VIII(ii)-b; and 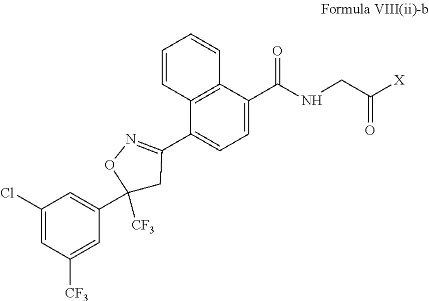
wherein, X is Cl or F,
f) reacting the compound of Formula VII-b or VIII(i)-b or VIII(ii)-b with the compound of Formula IX; 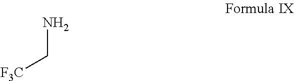
to obtain the compound of formula I-b.

15. A process for the preparation of compound of Formula I-b (Afoxolaner), said process comprising the steps of:
a) reacting the compound of Formula IV-b 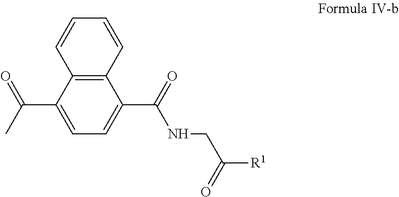
wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
with the compound of Formula IX 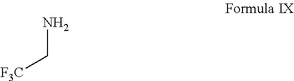
to obtain a compound of Formula X-b, 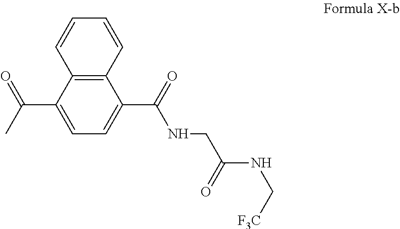 [SMILES: CC(=O)c1ccc(C(=O)NCC(=O)NCC(F)(F)F)c2ccccc12]
b) reacting the compound of Formula X-b with the compound of Formula V-b 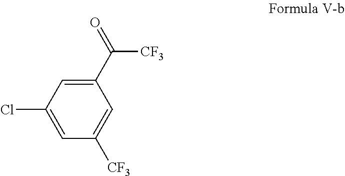 [SMILES: O=C(c1cc(Cl)cc(C(F)(F)F)c1)C(F)(F)F]
to obtain a compound of Formula XI(i)-b which is converted to a compound of Formula XI(ii)-b; 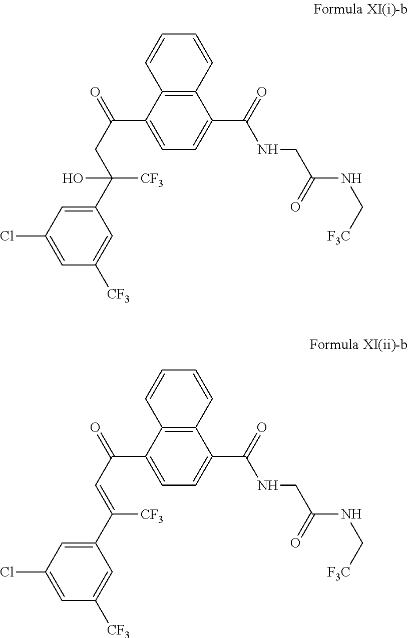 [SMILES: O=C(CNC(=O)c1ccc(C(=O)CC(O)(c2cc(Cl)cc(C(F)(F)F)c2)C(F)(F)F)c2ccccc12)NCC(F)(F)F ; O=C(CNC(=O)c1ccc(C(=O)/C=C(/c2cc(Cl)cc(C(F)(F)F)c2)C(F)(F)F)c2ccccc12)NCC(F)(F)F]
c) converting the compound of Formula XI(ii)-b into the compound of Formula I-b.

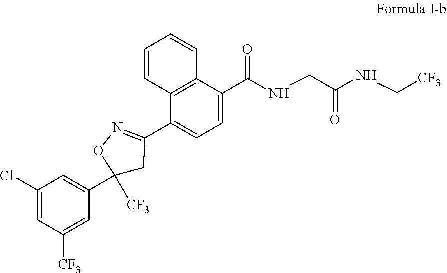 [SMILES: O=C(CNC(=O)c1ccc(C2=NOC(c3cc(Cl)cc(C(F)(F)F)c3)(C(F)(F)F)C2)c2ccccc12)NCC(F)(F)F]

16. The process as claimed in claims 14 and 15, wherein the compound of Formula II-b is prepared by a process comprising:
acylating the 1-fluro naphthalene to obtain a compound of Formula II-1-b, 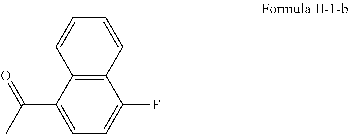
converting the compound of Formula II-1-b into a compound of Formula II-2-b using a cyano reagent, and 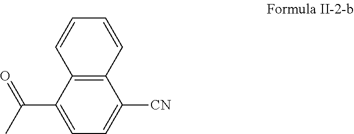
hydrolyzing the compound of Formula II-2-b and optionally, chlorinating using a chlorinating agent to obtain the compound of Formula II-b.

17. A compound of Formula IV-b; 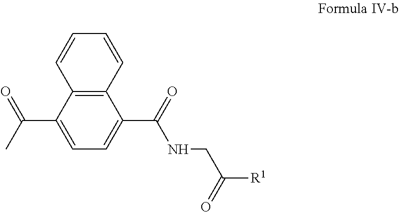
wherein, R¹ is selected from the group consisting of —OH, —OMe, —OEt, —O(n)Pr, —O(i)Pr, —O(n)Bu, —O(i)Bu, —O(sec)Bu, and —O(tert)Bu.
In another aspect, the present invention further provides the compound of Formula VI(i)-b & VI(ii)-b; 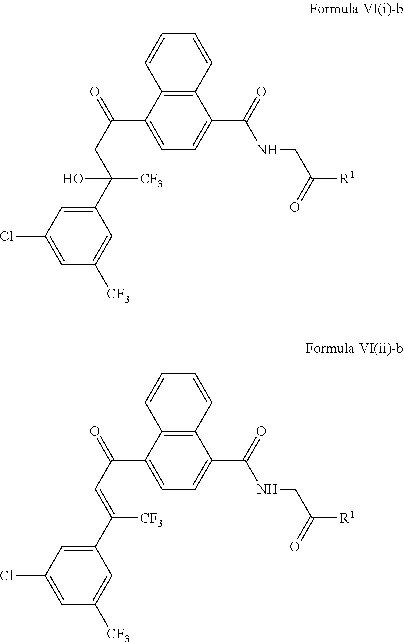
wherein, R¹ is selected from the group consisting of —OH, —OMe, —OEt, —O(n)Pr, —O(i)Pr, —O(n)Bu, —O(i)Bu, —O(sec)Bu, and —O(tert)Bu.

18. A compound of Formula VII-b 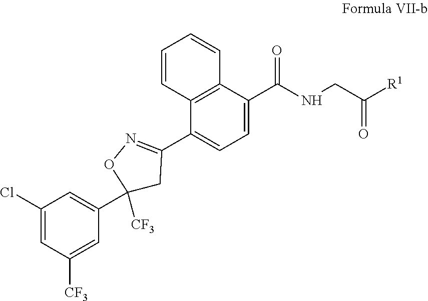
wherein, R¹ is selected from the group consisting of —OH, —OMe, —OEt, —O(n)Pr, —O(i)Pr, —O(n)Bu, —O(i)Bu, —O(sec)Bu, and —O(tert)Bu.

19. A process for the preparation of compound of Formula I-c (Lotilaner), 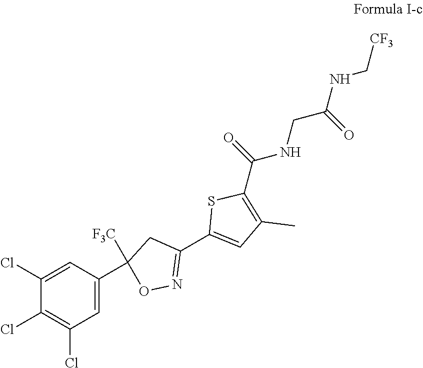 [SMILES: Cc1cc(C2=NOC(c3cc(Cl)c(Cl)c(Cl)c3)(C(F)(F)F)C2)sc1C(=O)NCC(=O)NCC(F)(F)F]
said process comprising the following steps:
a) reacting a compound of Formula II-c 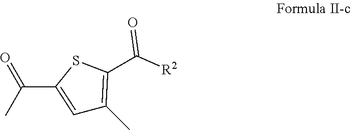
wherein, R² is OH, F, Cl or OR³&R³ is straight or branched chain C₁-C₄ alkyl, with a compound of Formula III 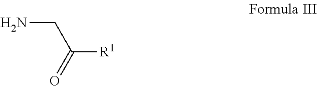
wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
to obtain a compound of Formula IV-c; 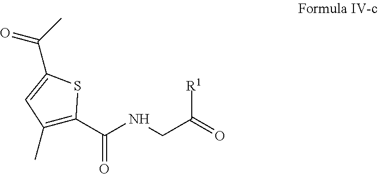
wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
b) reacting the compound of Formula IV-c with a compound of Formula V-c 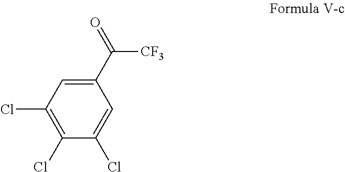 [SMILES: O=C(c1cc(Cl)c(Cl)c(Cl)c1)C(F)(F)F]
to obtain a compound of Formula VI(i)-c which is converted into a compound of Formula VI(ii)-c; 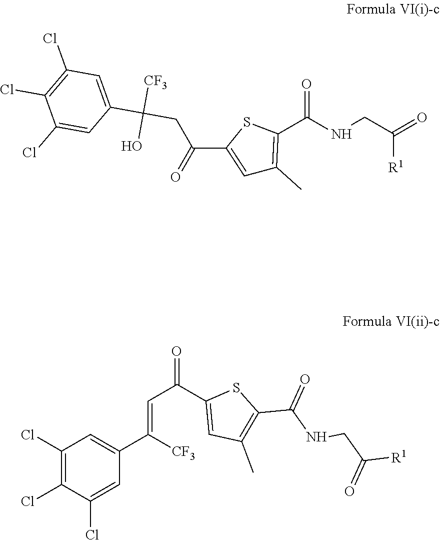
wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
c) converting the compound of Formula VI(ii)-c into a compound of Formula VII-c; 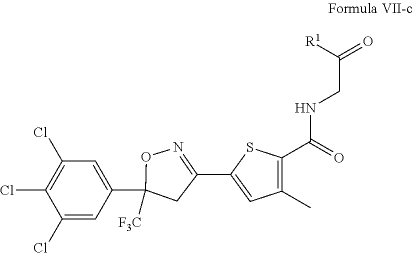
wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
d) converting the compound of Formula VII-c 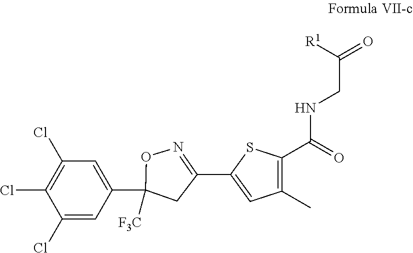
wherein, R¹ is selected from the group consisting of —OMe, —OEt, —O(n)Pr, —O(i)Pr, —O(n)Bu, —O(i)Bu, —O(sec)Bu, and —O(tert)Bu,
into a compound of Formula VIII(i)-c; 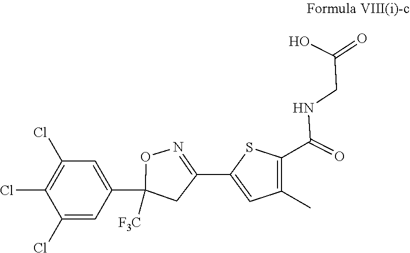 [SMILES: Cc1cc(C2=NOC(c3cc(Cl)c(Cl)c(Cl)c3)(C(F)(F)F)C2)sc1C(=O)NCC(=O)O]
e) optionally, converting the compound of Formula VIII(i)-c into a compound of Formula VIII(ii)-c; and 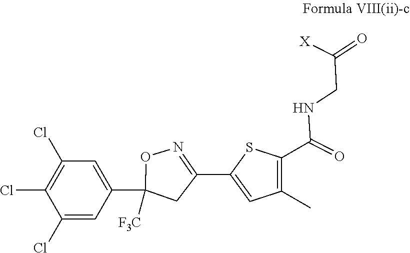
wherein, X is Cl or F,
f) reacting the compound of Formula VII-c or VIII(i)-c or VIII(ii)-c with the compound of Formula IX 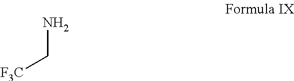
to obtain the compound of formula I-c.

20. A process for the preparation of a compound of Formula I-c, said process comprising the steps of:
a) reacting the compound of Formula IV-c 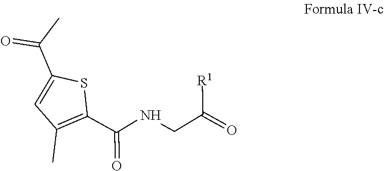
wherein, R¹ is selected from the group consisting of —OH, —OMe, —OEt, —O(n)Pr, —O(i)Pr, —O(n)Bu, —O(i)Bu, —O(sec)Bu, and —O(tert)Bu,
with the compound of Formula IX 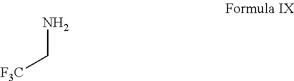
to obtain a compound of Formula X-c, 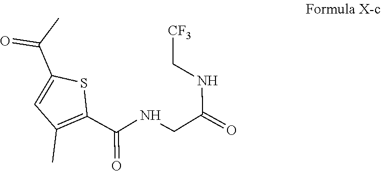 [SMILES: CC(=O)c1cc(C)c(C(=O)NCC(=O)NCC(F)(F)F)s1]
b) reacting the compound of Formula X-c with the compound of Formula V-c 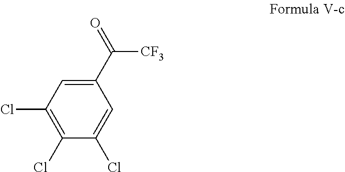 [SMILES: O=C(c1cc(Cl)c(Cl)c(Cl)c1)C(F)(F)F]
to obtain a compound of Formula XI(i)-c which is converted to a compound of Formula XI(ii)-c; 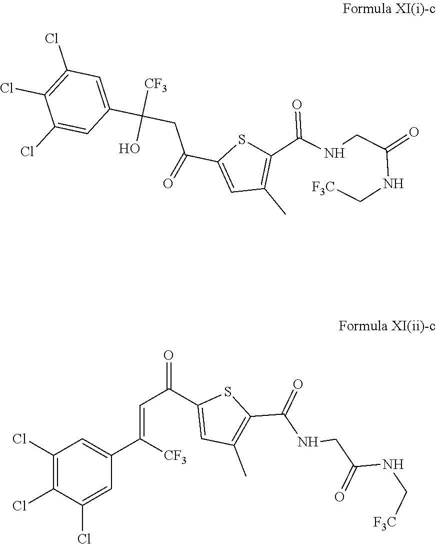 [SMILES: Cc1cc(C(=O)CC(O)(c2cc(Cl)c(Cl)c(Cl)c2)C(F)(F)F)sc1C(=O)NCC(=O)NCC(F)(F)F ; Cc1cc(C(=O)/C=C(/c2cc(Cl)c(Cl)c(Cl)c2)C(F)(F)F)sc1C(=O)NCC(=O)NCC(F)(F)F]
c) converting the compound of Formula XI(ii)-c into the compound of Formula I-c.

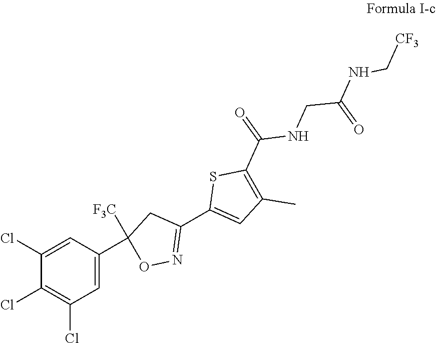 [SMILES: Cc1cc(C2=NOC(c3cc(Cl)c(Cl)c(Cl)c3)(C(F)(F)F)C2)sc1C(=O)NCC(=O)NCC(F)(F)F]

21. The process as claimed in claim 19, wherein the compound of Formula II-c is prepared by a process comprising:
acylating 2-fluoro-3-methylthiophene to obtain a compound of Formula II-1-c, 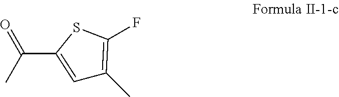
converting the compound of Formula II-1-c into a compound of Formula II-2-c using a cyano reagent, and 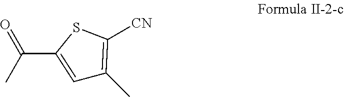
hydrolyzing the compound of Formula II-2-c and optionally, chlorinating using a chlorinating agent to obtain the compound of Formula II-c.

22. A compound of Formula IV-c 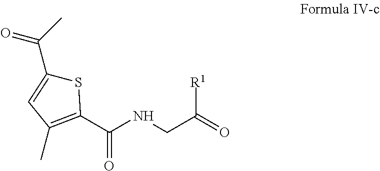
wherein, R¹ is selected from the group consisting of —OH, —OMe, —OEt, —O(n)Pr, —O(i)Pr, —O(n)Bu, —O(i)Bu, —O(sec)Bu, and —O(tert)Bu.

23. A compound of Formula VI(i)-c&VI(ii)-c; 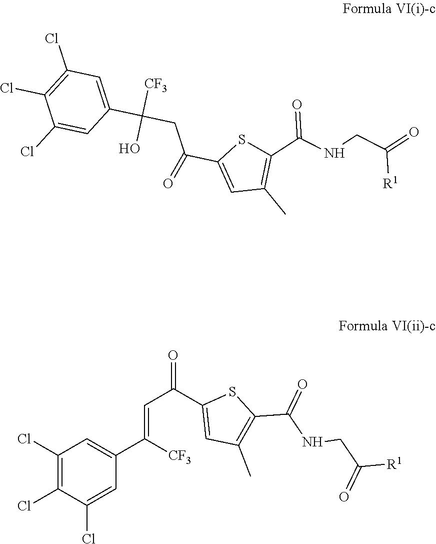
wherein, R¹ is selected from the group consisting of —OH, —OMe, —OEt, —O(n)Pr, —O(i)Pr, —O(n)Bu, —O(i)Bu, —O(sec)Bu, and —O(tert)Bu.

24. A compound of Formula VII-c 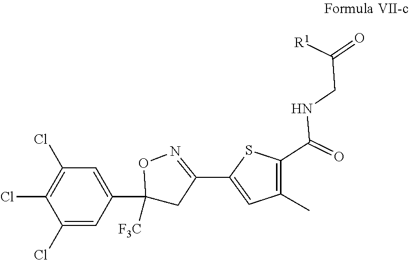
wherein, R¹ is selected from the group consisting of OH, —OEt, —O(n)Pr, —O(i)Pr, —O(n)Bu, —O(i)Bu, —O(sec)Bu, and —O(tert)Bu.

25. A compound of Formula X-c.

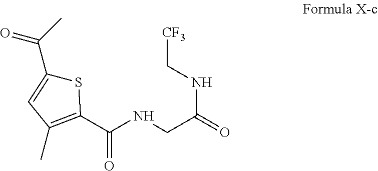 [SMILES: CC(=O)c1cc(C)c(C(=O)NCC(=O)NCC(F)(F)F)s1]

26. A compound of Formula XI(i)-c.

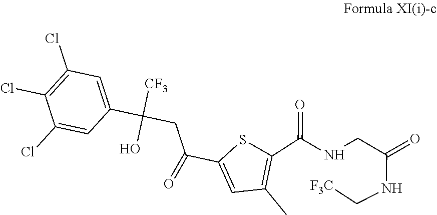 [SMILES: Cc1cc(C(=O)CC(O)(c2cc(Cl)c(Cl)c(Cl)c2)C(F)(F)F)sc1C(=O)NCC(=O)NCC(F)(F)F]

27. A compound of Formula XI(ii)-c. 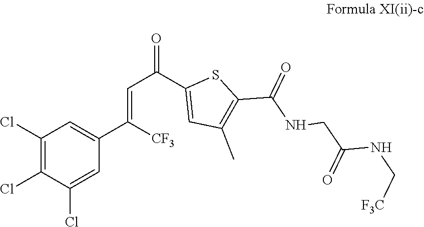 [SMILES: Cc1cc(C(=O)/C=C(/c2cc(Cl)c(Cl)c(Cl)c2)C(F)(F)F)sc1C(=O)NCC(=O)NCC(F)(F)F]
